(19) United States
(12) Patent Application Publication
(10) Pub. No.: US 2008/0234254 A1
(43) Pub. Date: Sep. 25, 2008

(54) Inhibitors of Histone Deacetylase

(75) Inventors: Ronald Grigg, Leeds (GB); Andrew Cook, Leeds (GB)

Correspondence Address: MORGAN LEWIS & BOCKIUS LLP, 1111 PENNSYLVANIA AVENUE NW, WASHINGTON, DC, 20004, US

(73) Assignee: UNIVERSITY OF LEEDS, Leeds (GB)

(21) Appl. No.: 11/628,992

(22) PCT Filed: Jun. 7, 2005
(86) PCT No.: PCT/GB05/02234

§ 371 Date: May 20, 2008

Related U.S. Application Data
(60) Provisional application No. 60/578,915, filed on Jun. 10, 2004.

(30) Foreign Application Priority Data
Jun. 10, 2004 (GB) 0412964.9

(51) Int. Cl. A61K 31/551; C07D 295/00; A61K 31/5375; C07C 237/00; A61K 31/164; C07D 213/00; A61K 31/44; C07D 217/00; A61K 31/47; C07D 401/02; A61K 31/496; C07D 471/02; A61K 31/437; A61K 31/495; C07D 239/24; A61K 31/505; C07D 233/54; A61K 31/4164; A61P 35/00; C07D 279/18; A61K 31/5415; C07D 219/00; A61K 31/473; C07D 243/10

(52) U.S. Cl. 514/220; 544/168; 514/237.8; 564/164; 514/620; 546/337; 514/357; 546/146; 514/307; 544/360; 514/253.01; 546/85; 514/292; 544/400; 514/252.12; 544/301; 514/274; 548/316.7; 514/392; 544/38; 514/226.2; 546/103; 514/297; 540/498

(57) ABSTRACT

The present invention relates to a benzamide derivative comprising a head, spacer and cap group wherein the spacer includes a benzene ring substituted with an additional spacer and wherein the additional spacer is an unsaturated group.

The present invention relates to benzamide derivatives, their use in the inhibition of histone deacetylase (HDAC) activity and their use in medicine in particular in the treatment of cancers such as haemotologic cancers. The invention also provides processes for the manufacture of the benzamide derivatives of the invention. In the eukaryotic cell, DNA is compacted to prevent transcription factor accessibility. When the cell is activated this compact DNA is made available to DNA-binding proteins, thereby allowing the induction of gene transcription (Beato, M., J. Med. Chem., 74, 711-724 (1996); Wolffe, A. P., Nature, 387, 16-17 (1997)). Nuclear DNA associates with histones to form a complex known as chromatin. The core histones, termed H2A, H2B, H3 and H4 surrounded by 146 base pairs of DNA form the fundamental unit of chromatin, the nucleosome. The N-terminal tails of the core histones contain lysines that are sites for post-transcriptional acetylation. Acetylation neutralizes the potential of the side chain to form a positive charge on the lysine side chain, and is thought to impact chromatin structure.

Histone deacetylases (HDACs) are zinc-containing enzymes which catalyse the removal of acetyl groups from the ε-amino termini of lysine residues clustered near the amino terminus of nucleosomal histones. HDACs may be divided into three classes, the first (HDAC 1, 2, 3 and 8) represented by yeast Rpd3-like proteins, the second (HDAC 4, 5, 6, 7, 9 and 10) represented by yeast Hdal-like proteins and a third class of NAD+ dependent HDACs. Deregulation of certain HDAC inhibitors has been associated with several cancers and HDAC inhibitors, such as Trichostatin A (a natural product isolated from Streptomyces hygroscopicus), have been shown to exhibit significant anti-tumour effects and inhibition of cell-growth (Meinke, P. T., Current Medicinal Chemistry, 8, 211-235 (2001)). Yoshida et al, Exper. Cell Res., 177, 122-131 (1988) teaches that Trichostatin A causes arrest of rat fibroblasts at the G1 and G2 phases of the cell cycle, thereby implicating HDAC in cell cycle regulation. Furthermore, Trichostatin A has been shown to induce terminal differentiation, inhibit cell growth, and prevent the formation of tumours in mice (Finnin et al., Nature, 401, 188-193 (1999)).

Elucidation of the crystal structure of the HDAC inhibitor, Trichostatin A, revealed an active site consisting of a tubular pocket, a zinc-binding site and two Asp-His charge-relay systems all of which are believed to be involved in the mechanism of HDAC inhibition (Finnin et al., Nature, 401, 188-193 (1999). The zinc binding region of the molecule can be defined as the “head” group. HDAC inhibitors such as Trichostatin A are benzamide derivatives in which the benzamide group is referred to as the “head”. The aliphatic chain which allows the molecule to insert into the deep narrow pocket of the enzyme can be defined as a “spacer” group. The spacer group is typically a hydrophobic group. Since the pocket is 11 Angstrom in length, a spacer is likely to be less than 11 Angstrom, for example about 6 Angstrom (Finnin et al., Nature, 401, 188-193 (1999). A further group, the “cap” group, within the molecule is responsible for capping the pocket by making contact at the pocket entrance and in an adjacent groove (Finnin et al., Nature, 401, 188-193 (1999). The cap group is typically a bulky molecule, for example, one or more ringed systems for example aromatic groups such as the aromatic dimethylamino-phenyl group at the end of Trichostatin A (Finnin et al., Nature, 401, 188-193 (1999).

A number of potential HDAC inhibitors are known.

Thus, International Patent Application No. WO 03/087057 describes substituted phenyl benzamide derivatives useful as HDAC inhibitors.

Similarly, WO 03/092686 describes benzamide compounds which are provided with a heterocyclyl phenyl or heterocyclyl thiophenyl derivatives, eg thiophene, thiazole and thiadiazole derivatives.

Moreover, the compound MS275, described in Japanese patent applications 10152462 and 11302173, U.S. Pat. No. 6,174,905B1 and European patent application 847992A1, is known and currently undergoing clinical trials for the treatment of patients suffering from, inter alia, leukaemia and haematologic cancer in general.

Without wishing to be bound by theory, known HDAC inhibitors contain groups that may be analogous to head, cap and spacer groups. MS275 is a benzamide derivative comprising head, spacer and capping groupings. More specifically MS275 is N-(2-aminophenyl)-4-[N-pyridin-3-yl-methoxycarbonyl)aminomethyl]benzamide (I).

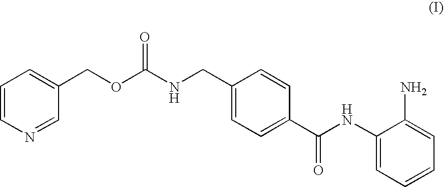

The spacer group in MS275 is a substituted benzene ring. The spacer group can be seen as the central substituted benzene ring within formula (I).

The present inventors have now surprisingly identified a group of compounds which comprise alternative spacer moieties to that in MS275. Such compounds have significantly improved activity over known HDAC inhibitors, including MS275.

According to a first aspect of the invention there is provided a benzamide derivative comprising a head, spacer and cap group wherein the spacer includes a benzene ring substituted with an additional spacer and wherein the additional spacer is an unsaturated group.

Preferably, the additional spacer is a vinyl substituent. Preferably still the additional spacer is a propylene derivative.

Preferably, the spacer is a propylene substituted benzene ring.

Preferably the head and cap groups are as defined herein.

According to a second aspect of the invention, there is provided a compound of formula (II);

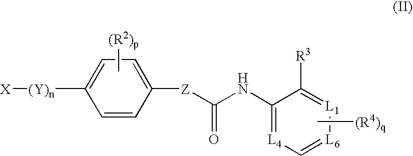

wherein:
the group X hereinafter referred to as the CAP group is a compound of general formula (III) or (IV);

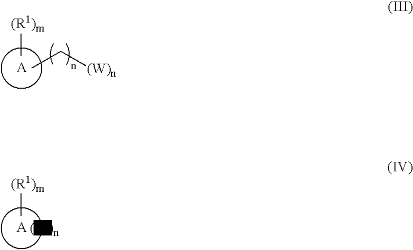

W is carbon, —CH—, —CH₂—, nitrogen, sulphur, oxygen, —N(R^{a})—, —C(O)O—, —C(O)—, —N(R^{a})C(O)—, —N(R^{a})C(O)N(R^{b})—, —N(R^{a})C(O)O—, —OC(O)N(R^{a})—, —C(O)N(R^{a})—, S(O)ᵣ—, —SO₂N(R^{a})—, —N(R^{a})SO₂—, —N(R^{a})C(S)N(R^{b})—, —N(R^{a})C(S)O—, —C(S)— or —C(S)N(R^{a})—; wherein R^{a} and R^{b} are independently selected from hydrogen or C₁₋₆alkyl optionally substituted by one or more V and r is 0-2; and wherein W is optionally substituted by a compound of formula (III) or (IV);
n is 1, 2, 3, 4, 5 or 6;
Ring A is an optionally substituted carbocyclyl or heterocyclyl group wherein each substitutable carbon or heteroatom in Ring A is optionally and independently substituted by one or more of halo, C₁₋₆ alkyl, carbocyclyl or heterocyclyl and wherein if Ring A contains an —NH— moiety other than W that nitrogen may be optionally substituted by a group selected from K;
L is carbon or nitrogen;
R¹ is a substituent on carbon, including carbon in the optional substituents in Ring A, and is selected from oxygen, halo, nitro, cyano, hydroxy, trifluoromethyl, trifluoromethoxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N—(C₁₋₆alkyl)amino, N,N—(C₁₋₆alkyl)₂-amino, C₁₋₆alkanoylamino, N—(C₁₋₆alkyl)carbamoyl, N,N—(C₁₋₆alkyl)₂-carbamoyl, C₃₋₈cycloalkyl, C₃₋₈cycloalkylC₁₋₆alkyl, C₁₋₆alkylS(O)ₐ wherein a is 0 to 2, C₁₋₆alkoxycarbonyl, N—(C₁₋₆alkyl)sulphamoyl, N,N—(C₁₋₆alkyl)₂sulphamoyl, aryl, aryloxy, arylC₁₋₆alkyl, heterocyclic group, (heterocyclic group)C₁₋₆alkyl, or a group (D-E-); wherein R¹, including group (D-E-), may be optionally substituted on carbon by one or more T; and wherein if said heterocyclic group contains an —NH— moiety that nitrogen may be optionally substituted by J;
T is halo, nitro, cyano, hydroxy, trifluoromethyl, trifluoromethoxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N—(C₁₋₆alkyl)amino, N,N—(C₁₋₆alkyl)₂amino, C₁₋₆alkanoylamino, N—(C₁₋₆alkyl)carbamoyl, N,N—(C₁₋₆alkyl)₂carbamoyl, C₁₋₆alkylS(O)ₐ wherein a is 0 to 2, C₁₋₆alkoxycarbonyl, N—(C₁₋₆alkyl)sulphamoyl, N,N—(C₁₋₆alkyl)₂sulphamoyl, or a group (D′-E′-); wherein T, including group (D′-E′-), may be optionally substituted on carbon by one or more R;
R and Q are independently selected from halo, nitro, cyano, hydroxy, trifluoromethyl, trifluoromethoxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N—(C₁₋₆alkyl)amino, N,N—(C₁₋₆alkyl)₂amino, C₁₋₆alkanoylamino, N—(C₁₋₆alkyl)carbamoyl, N,N—(C₁₋₆alkyl)₂carbamoyl, C₁₋₆alkylS(O)ₐ wherein a is 0 to 2, C₁₋₆alkoxycarbonyl, N—(C₁₋₆alkyl)sulphamoyl, N,N—(C₁₋₆alkyl)₂sulphamoyl;
G, J and K are independently selected from C₁₋₈alkyl, C₂₋₈alkenyl, C₂₋₈alkynyl, C₁₋₆alkanoyl, C₁₋₈alkylsulphonyl, C₁₋₈alkoxycarbonyl, carbamoyl, N—(C₁₋₈alkyl)carbamoyl, N,N—(C₁₋₈alkyl)₂carbamoyl, benzyloxycarbonyl, benzoyl, phenylsulphonyl, aryl, arylC₁₋₆alkyl or (heterocyclic group)C₁₋₆alkyl; wherein G, J and K may be optionally substituted on carbon by one or more P; and wherein if said heterocyclic group contains an —NH— moiety that nitrogen may be optionally substituted by hydrogen or C₁₋₆alkyl;
P is halo, nitro, cyano, hydroxy, trifluoromethyl, trifluoromethoxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N—(C₁₋₆alkyl)amino, N,N—(C₁₋₆alkyl)₂amino, C₁₋₆alkanoylamino, N—(C₁₋₆alkyl)carbamoyl, N,N—(C₁₋₆alkyl)₂carbamoyl, C₁₋₆alkylS(O)ₐ wherein a is 0 to 2, C₁₋₆alkoxycarbonyl, C₁₋₆alkoxycarbonylamino, N—(C₁₋₆alkyl)sulphamoyl, N,N—(C₁₋₆alkyl)₂sulphamoyl, aryl, aryloxy, arylC₁₋₆alkyl, arylC₁₋₆alkoxy, heterocyclic group, (heterocyclic group)C₁₋₆alkyl, (heterocyclic group)C₁₋₆alkoxy, or a group (D″-E″-); wherein P, including group (D″-E″-), may be optionally substituted on carbon by one or more Q;
D, D′ and D″ are independently selected from C₁₋₆alkyl, C₂₋₆alkenyl, C₂₋₆alkynyl, C₃₋₈cycloalkyl, C₃₋₈cycloalkylC₁₋₆alkyl, aryl, arylC₁₋₆alkyl, heterocyclic group, (heterocyclic group)C₁₋₆alkyl, phenyl or phenylC₁₋₆alkyl wherein D, D′ and D″ may be optionally substituted on carbon by one or more M; and wherein if said heterocyclic group contains an —NH— moiety that nitrogen may be optionally substituted by a group selected from G;
E, E′ and E″ are independently selected from —N(R^{a})—, —O—, —C(O)O—, —C(O)—, —N(R^{a})C(O)—, —N(R^{a})C(O)N(R^{b})—, —N(R^{a})C(O)O—, —OC(O)N(R^{a})—, —C(O)N(R^{a})—, S(O)ᵣ—, —SO₂N(R^{a})—, —N(R^{a})SO₂—; wherein R^{a} and R^{b} are independently selected from hydrogen or C₁₋₆alkyl optionally substituted by one or more V and r is 0-2;
M and V are independently selected from halo, nitro, cyano, hydroxy, trifluoromethyl, trifluoromethoxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N—(C₁₋₆alkyl)amino, N,N—(C₁₋₆alkyl)₂amino, C₁₋₆alkanoylamino, N—(C₁₋₆alkyl)carbamoyl, N,N—(C₁₋₆alkyl)₂carbamoyl, C₁₋₆alkylS(O)ₐ wherein a is 0 to 2, C₁₋₆alkoxycarbonyl, N—(C₁₋₆alkyl)sulphamoyl or N,N—(C₁₋₆alkyl)₂sulphamoyl;
m is 0, 1, 2, 3 or 4; wherein the values of R¹ may be the same or different;
wherein the group Y, hereinafter referred to as the ADDITIONAL SPACER group, is an unsaturated group;
R² is absent or halo;
p is 0, 1 or 2; wherein the values of R² are the same or different;
Z is absent, a direct carbon-carbon bond, C₁₋₆alkyl, C₂₋₆alkenyl, C₂₋₆alkynyl or —C(R^{c})═N—O—; wherein R^{c} is independently selected from hydrogen or C₁₋₆alkyl optionally substituted by one or more V and r is 0-2;
R³ is absent, amino or hydroxy;
R⁴ is halo, nitro, cyano, hydroxy, trifluoromethyl, trifluoromethoxy, amino, carboxy, carbamoyl, mercapto, sulphamoyl, C₁₋₃alkyl, C₂₋₃alkenyl, C₂₋₃alkynyl, C₁₋₃alkoxy, C₁₋₃alkanoyl, C₁₋₃alkanoyloxy, N—(C₁₋₃alkyl)amino, N,N—(C₁₋₃alkyl)₂amino, C₁₋₃alkanoylamino, N—(C₁₋₃alkyl)carbamoyl, N,N—(C₁₋₃alkyl)₂carbamoyl, C₁₋₃alkylS(O)ₐ wherein a is 0 to 2, C₁₋₃alkoxycarbonyl, N—(C₁₋₃alkyl)sulphamoyl or N,N—(C₁₋₃alkyl)₂sulphamoyl;
q is 0, 1 or 2; wherein the values of R⁴ are the same or different;
or a pharmaceutically acceptable salt or in vivo hydrolysable ester or amide thereof.

Preferably, L is nitrogen at only one of positions L₁, L₄ or L₆.

In a preferred aspect of the invention there is provided a compound of formula (IIb)

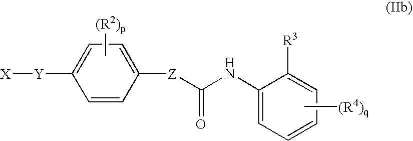

wherein X, Y, Z, R², R³, R⁴, p and q are as defined herein.

Preferably, Y is the 2-propylene derivative (V) or the optionally functionalised derivative of the double bond in (V) such as the epoxide, diol, or the reduced 2-propyl product (VI);

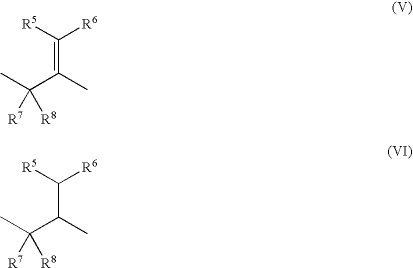

wherein R⁵, R⁶, R⁷ and R⁸ are independently selected from hydrogen, halo, C₁₋₆alkyl, C₃₋₈cycloalkyl, C₃₋₈cycloalkylC₁₋₆alkyl, aryl, arylC₁₋₆alkyl, heterocyclic group, (heterocyclic group)C₁₋₆alkyl, phenyl, phenylC₁₋₆alkyl, C₁₋₆alkoxy, C₁₋₆alkanoyl, C₁₋₆alkanoyloxy, N—(C₁₋₆alkyl)amino, N,N—(C₁₋₆alkyl)₂amino, C₁₋₆alkanoylamino, N—(C₁₋₆alkyl)carbamoyl, N,N—(C₁₋₆alkyl)₂carbamoyl, C₁₋₆alkylS(O)ₐ wherein a is 0 to 2, C₁₋₆alkoxycarbonyl, N—(C₁₋₆alkyl)sulphamoyl or N,N—(C₁₋₆alkyl)₂sulphamoyl.

Further values of Ring A are as follows. Such values may be used where appropriate with any of the definitions, claims or embodiments defined hereinbefore or hereinafter.

Ring A is an optionally and independently substituted aryl, arylC₁₋₆alkyl, heterocyclic group, (heterocyclic group)C₁₋₆alkyl, phenyl, phenylC₁₋₆alkyl, pyridyl, quinolyl, indolyl, pyrimidinyl, morpholinyl, piperidinyl, piperazinyl, pyridazinyl, pyrazinyl, thiazolyl, thienyl, thienopyrimidinyl, thienopyridinyl, purinyl, 1′,2′,3′,6′-tetrahydropyridinyl, triazinyl, oxazolyl, pyrazolyl, furanyl or tetrahydro-β-carbolinyl; wherein each substitutable carbon or heteroatom in Ring A is optionally and independently substituted by one or more of halo, C1-6 alkyl, carbocyclyl or heterocyclyl; and wherein if Ring A contains an —NH— moiety other than W that nitrogen may be optionally substituted by a group selected from K. Preferably, Ring

A is a substituted piperazinyl group, for example, a piperazinyl group in which a nitrogen is substituted by an aryl group, for example a halo substituted aryl group.

In this specification the term “alkyl” includes both straight and branched chain alkyl groups. For example “C₁₋₈alkyl” and “C₁₋₆alkyl” includes methyl, ethyl, propyl, isopropyl, pentyl, hexyl, heptyl and t-butyl. However, references to individual alkyl groups such as “propyl” are specific to the straight-chained version only and references to individual branched chain alkyl groups such as “isopropyl” are specific for the branched chain version only. The term “halo” refers to fluoro, chloro, bromo and iodo.

Where optional substituents are chosen from “one or more” groups it is to be understood that this definition includes all substituents being chosen from one of the specified groups or the substituents being chosen from two or more of the specified groups.

A “heterocyclyl” and “heterocyclic group” are saturated, partially saturated or unsaturated, mono-, bi- or tricyclic ring containing 3-18 atoms of which at least one atom (for example 2 or 3 atoms) is chosen from nitrogen, sulphur or oxygen, which may be carbon or nitrogen linked, wherein a CH₂ group can optionally be replaced by a C(O), wherein a ring sulphur atom may be optionally oxidised to form the S-oxide(s). Examples and suitable values of the term “heterocyclyl” and “heterocyclic group” are thiazolidinyl, pyrrolidinyl, 1,3-benzodioxolyl, 1,2,4-oxadiazolyl, 2-azabicyclo[2.2.1]heptyl, morpholinoyl, tetrahydrofuranyl, furanyl, tetrahydropyranyl, piperidinyl, piperazinyl, thiomorpholinyl, 1,3-dioxoanyl, homopiperazinyl, thienyl, pyrrolyl, pyrazolyl, oxodiazolyl, tetrazolyl, oxazolyl, thienopyrimidinyl, thienopyridinyl, thieno[3,2-d]pyrimidinyl, 1,3,5-triazinyl, purinyl, 1,2,3,4-tetrahydroquinolyl, 1,2,3,4-tetrahydroisoquinolyl, 1′,2′,3′,6′-tetrahydropyridinyl, tetrahydro-β-carbolinyl, tetrahydropyridinyl, benzimidazolyl, benzthiazolyl, benzoxazolyl, benzothienyl, benzofuranyl, indazolyl, quinazolinyl, cinnolinyl, phthalazinyl, quinoxalinyl, napthyridinyl, benzotriazolyl, pyrrolothienyl, imidazothienyl, isoxazolyl, imidazolyl), thiadiazolyl, isothiazolyl, 1,2,3-triazolyl, 1,2,4-triazolyl, pyranyl, indolyl, pyrimidinyl, thiazolyl, pyrazinyl, pyridazinyl, pyridyl, quinolyl, quinazolinyl and 1-isoquinolinyl.

An “aryl” group includes, for example, phenyl, indenyl, indanyl, naphthyl, tetrahydronaphthyl or fluorenyl.

An example of “C₁₋₆alkanoyloxy” is acetoxy. Examples of “C₁₋₈alkoxycarbonyl” and “C₁₋₆alkoxycarbonyl” include methoxycarbonyl, ethoxycarbonyl, n- and t-butoxycarbonyl. Examples of “C₂₋₆alkenyl” include vinyl, allyl and 1-propenyl. Examples of “C₂₋₆alkynyl” are ethynyl and 2-propynyl. Examples of “C₁₋₆alkoxy” include methoxy, ethoxy, propoxy and t-butoxy. Examples of “C₁₋₆alkanoylamino” include formamido, acetamido and propionylamido. Examples of “C₁₋₆alkylS(O)ₐ wherein a is 0 to 2” include methylthio, ethylthio, methylsulphinyl, ethylsulphinyl, mesyl and ethylsulphonyl. Examples of “C₁₋₆alkanoyl” include propionyl and acetyl. Examples of “N—(C₁₋₆alkyl)amino” include methylamino, ethylamino, propylamino and butylamino. Examples of “N,N—(C₁₋₆alkyl)₂amino include di-N-methylamino, di-(N-ethyl)amino and N-ethyl-N-methyl-amino.

Examples of “N—(C₁₋₆alkyl)sulphamoyl” are N-(methyl)sulphamoyl and N-(ethyl)-sulphamoyl. Examples of “N,N—(C₁₋₆alkyl)₂sulphamoyl” are N,N-(dimethyl)sulphamoyl and N-methyl-N-ethyl-sulphamoyl. Examples of “N—(C₁₋₆alkyl)carbamoyl” are methylaminocarbonyl and ethylaminocarbonyl. Examples of “N,N—(C₁₋₆alkyl)₂ carbonyl” are dimethylaminocarbonyl and methylethylamino carbonyl. Examples of “(heterocyclic group)C₁₋₆alkyl” include piperidin-1-ylmethyl, piperidin-1-ylethyl, piperidin-1-ylpropyl, pyridylmethyl, 3-morpholinopropyl, 2-morpholinoethyl and 2-pyrimid-2-ylethyl. Examples of “arylC₁₋₆alkyl” include benzyl, 2-phenylethyl, 2-phenylpropyl and 3-phenylpropyl. Examples of “aryloxy” include phenoxy and naphthyloxy. Examples of “C₃₋₈cycloalkyl” include cyclopropyl and cyclohexyl. Examples of “C₃₋₈cycloalkylC₁₋₆alkyl” include cyclopropylmethyl and 2-cyclohexylmethyl.

Within this specification composite terms are used to describe groups comprising more than one functionality such as arylC₁₋₆alkyl. Such terms are to be interpreted as is understood by a person skilled in the art.

In a further preferred aspect of the invention there is provided a compound of formula (II) or (IIb) wherein the number of W groups in the compounds of general formula (III) or (IV) is 1.

A suitable pharmaceutically acceptable salt of a compound of the invention is, for example, an acid-addition salt of a compound of the invention which is sufficiently basic; for example, an acid-addition salt with, for example, an inorganic or organic acid, for example, acetic acid, hydrochloric, hydrobromic, sulphuric, phosphoric, trifluoroacetic, citric or maleic acid.

In addition a suitable pharmaceutically acceptable salt of a compound of the invention which is sufficiently acidic is an alkali metal salt, for example a sodium or potassium salt, an alkaline earth metal salt, for example a calcium or magnesium salt, an ammonium salt or a salt with an organic base which affords a physiologically-acceptable cation, for example a salt with methylamine, dimethylamine, trimethylamine, piperidine or morpholine.

The compounds of formula (II) may be administered in the form of an in vivo hydrolysable ester or in vivo hydrolysable amide of a compound of the formula (II).

An in vivo hydrolysable ester of a compound of the formula (II) containing a hydroxy group includes inorganic esters such as phosphate esters and acyloxyalkyl ethers and related compounds which as a result of in vivo hydrolysis of the ester break down to give the parent hydroxy group. Examples of acyloxyalkyl ethers include acetoxymethoxy and 2,2-dimethylpropionyloxy-methoxy. A selection of in vivo hydrolysable ester forming groups for hydroxy include alkanoyl, benzoyl, phenylacetyl and substituted benzoyl and phenylacetyl, alkoxycarbonyl (to give alkyl carbonate esters). Dialkylcarbamoyl and N—(N,N-dialkylaminoethyl)-N-alkylcarbamoyl (to give carbamates), N,N-dialkylaminoacetyl and carboxyacetyl. Examples of substituents on benzoyl include morpholino and piperazino.

A suitable value for an in vivo hydrolysable amide of a compound of the formula (II) containing a carboxy group is, for example, N—C₁₋₆alkyl amide or N,N-di-C₁₋₆alkyl amide such as N-methyl, N-ethyl, N-propyl, N,N-dimethyl, N-ethyl-N-methyl or N,N-diethyl amide.

Some compounds of the formula (II) may have chiral centres and/or geometric isomeric centres (E- and Z-isomers), and it is to be understood that the invention encompasses all such optical, diastereomers and geometric isomers that possess HDAC inhibitory activity.

The invention also includes prodrugs for the active pharmaceutical species of the described compounds, for example in which one or more functional groups are protected or derivatised but can be converted in vivo to the functional group, as in the case of esters of carboxylic acids convertible in vivo to the free acid, or in the case of protected amines, to the free amino group. The term “prodrug,” as used herein, represents in particular compounds which are rapidly transformed in vivo to the parent compound, for example, by hydrolysis in blood. A thorough discussion is provided in T. Higuchi and V. Stella, Pro-drugs as Novel Delivery Systems, Vol. 14 of the A.C.S. Symposium Series, Edward B. Roche, ed., Bioreversible Carriers in Drug Design, American Pharmaceutical Association and Pergamon Press, 1987; H Bundgaard, ed, Design of Prodrugs, Elsevier, 1985; and Judkins, et al. Synthetic Communications, 26(23), 4351-4367 (1996), each of which is incorporated herein by reference.

Thus preferred compounds which may be mentioned include those selected from the group;

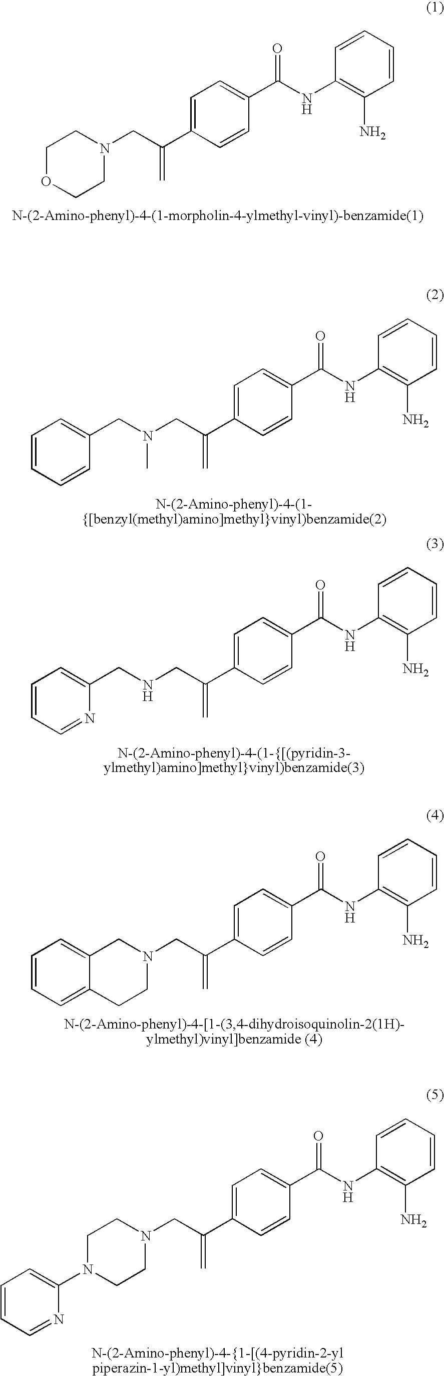

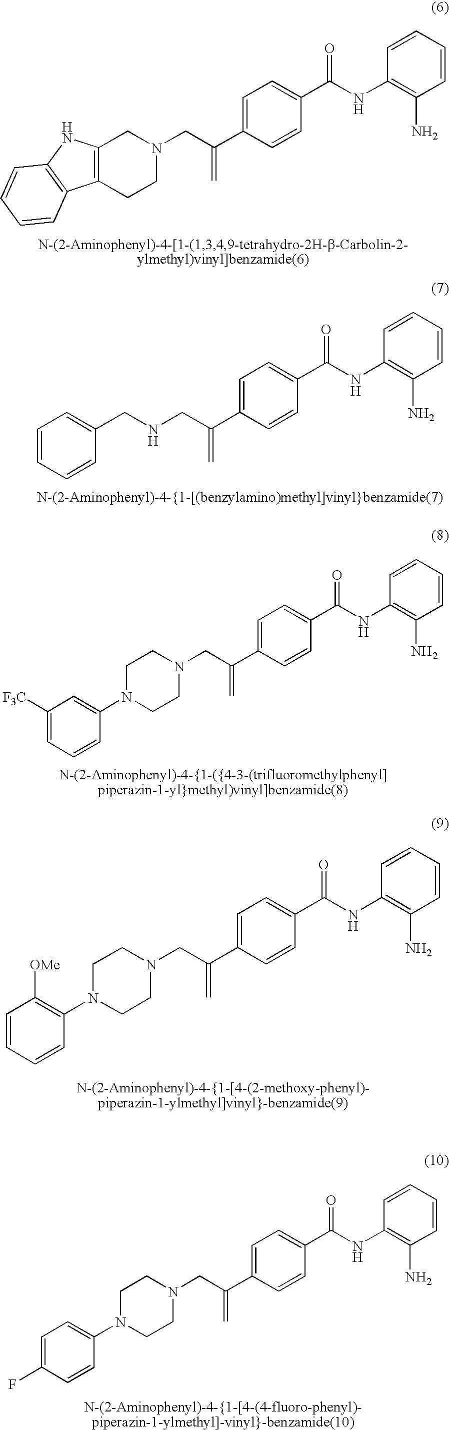

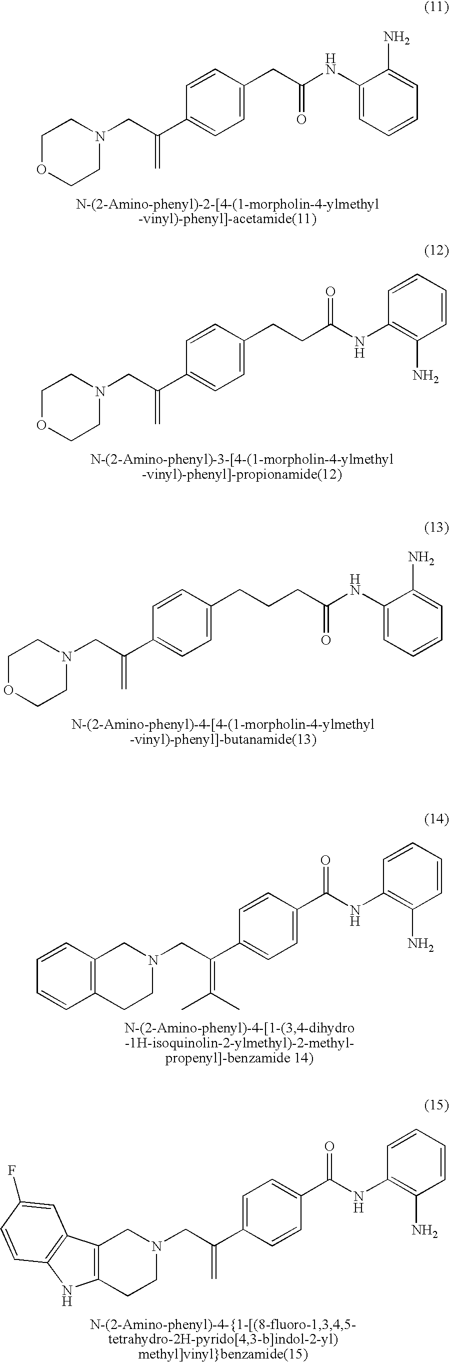

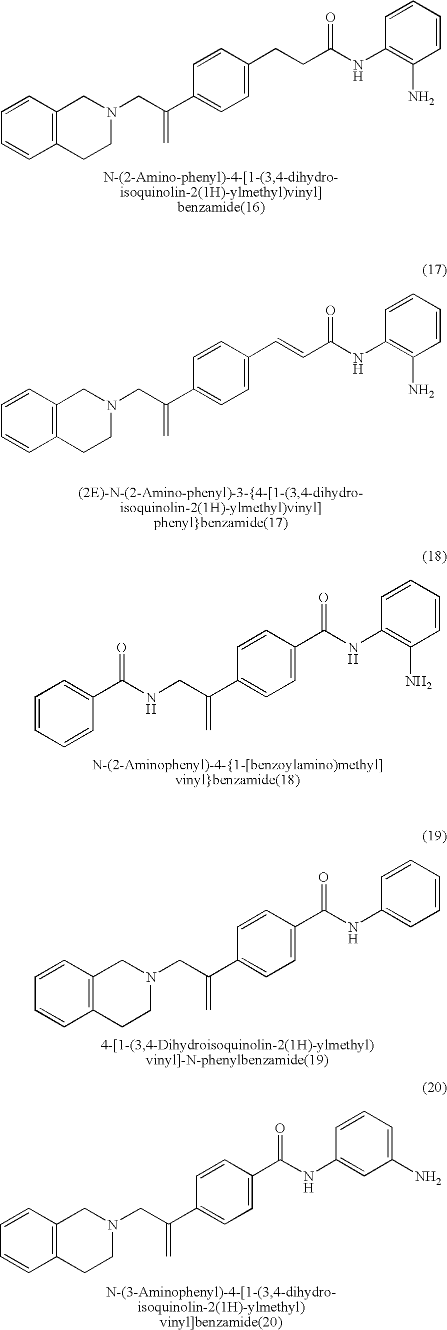

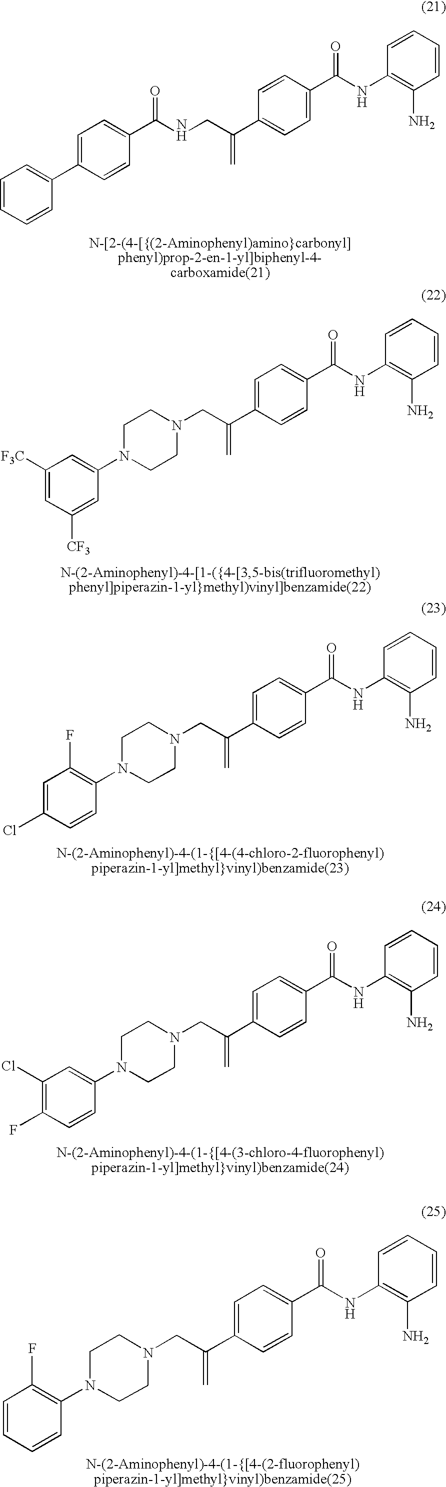

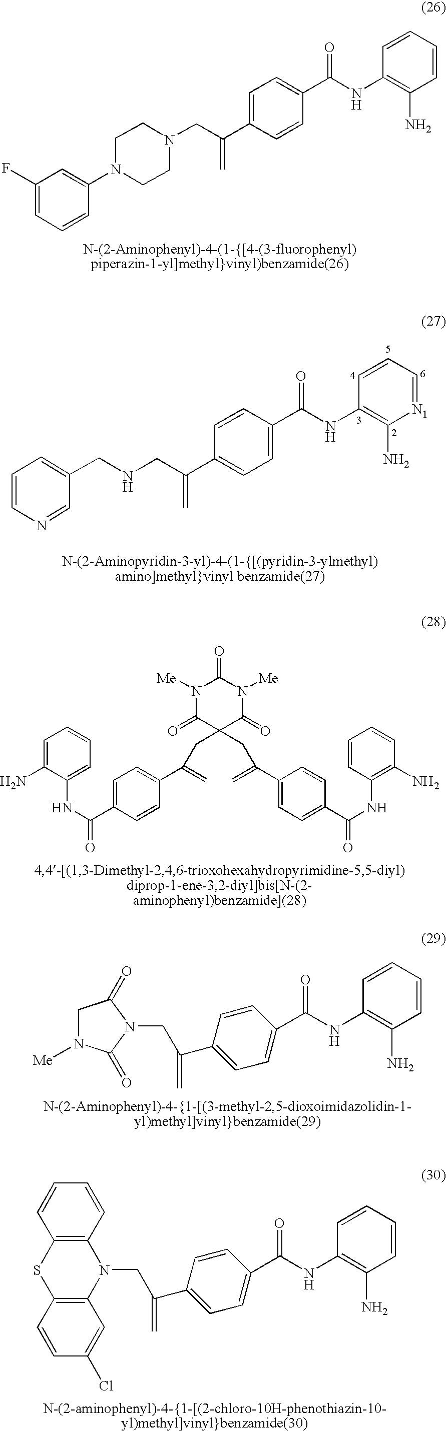

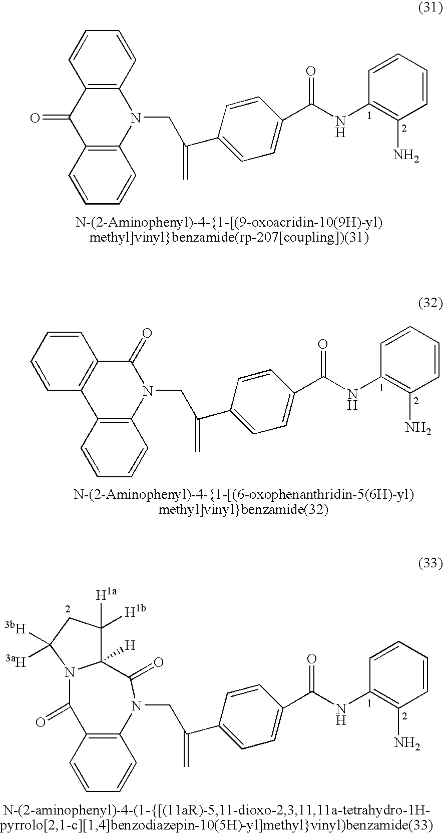

Preferred compounds are N-(2-amino-phenyl)-4-[1-(3,4-dihydroisoquinolin-2(1H)-ylmethyl)vinyl]benzamide (4), N-(2-aminophenyl)-4-[1-(1,3,4,9-tetrahydro-2H-β-carbolin-2-ylmethyl)vinyl]benzamide (6) and N-(2-aminophenyl)-4-{1-({4-3-(trifluoromethylphenyl]piperazin-1-yl}methyl)vinyl]benzamide (8).

The compounds of the invention may be referred to as benzamide derivatives wherein the benzamide group (optionally substituted with an amine group) is herein referred to as the HEAD group.

The compounds and salts of the invention can be incorporated into pharmaceutical compositions. Such compositions typically include the compound or salt and a pharmaceutically acceptable carrier. As used herein the language “pharmaceutically acceptable carrier” includes solvents, dispersion media, coatings, antibacterial and antifungal agents, isotonic and absorption delaying agents, and the like, compatible with pharmaceutical administration. Supplementary active compounds can also be incorporated into the compositions.

A pharmaceutical composition is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash.

Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the compounds may be prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of compound calculated to produce the desired therapeutic effect in association with the required pharmaceutical carrier.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit high therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in therapy, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

Exemplary doses include milligram or microgram amounts of the compound per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram. It is furthermore understood that appropriate doses of a compound depend upon the potency of the compound with respect to the expression or activity to be modulated. When one or more of these compounds is to be administered to an animal (e.g., a human), a physician, veterinarian, or researcher may, for example, prescribe a relatively low dose at first, subsequently increasing the dose until an appropriate response is obtained. In addition, it is understood that the specific dose level for any particular animal subject will depend upon a variety of factors including the activity of the specific compound employed, the age, body weight, general health, gender, and diet of the subject, the time of administration, the route of administration, the rate of excretion, any drug combination, and the degree of expression or activity to be modulated.

According to a further aspect of the invention there is provided a process for the manufacture of a compound of formula II which comprises reacting the following compounds in the presence of a catalyst:
i) a nucleophile of formula (III) or (IV) wherein the nucleophilic group is either W or is located within R¹;
ii) allene gas or a substituted allene; and
iii) a halogen or triflate substituted aryl molecule of formula (VII) wherein:

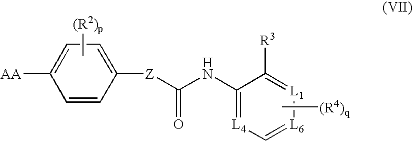

AA is independently selected from halo or triflate; and
Z, R², R³, R⁴, L, p and q are as hereinbefore defined.

In a preferred process of the invention, the halogen or triflate substituted aryl molecule in (iii) is of formula (VIIb) wherein:

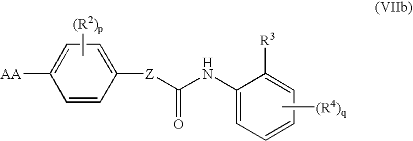

AA is independently selected from halo or triflate; and
Z, R², R³, R⁴, p and q are as hereinbefore defined.

According to a further aspect of the invention there is provided a process for the manufacture of a compound of formula II which comprises the steps of

a) reacting the following compounds in the presence of a catalyst:
i) a nucleophile of formula (III) or (IV) wherein the nucleophilic group is either W or is located within R¹;
ii) allene gas or a substituted allene; and
iii) a halogen or triflate substituted aryl molecule of formula (IX)

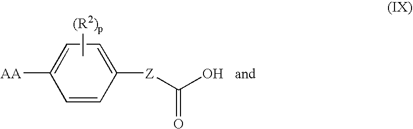

b) reacting the product of (a) with a compound of formula (X) in the presence of a coupling reagent

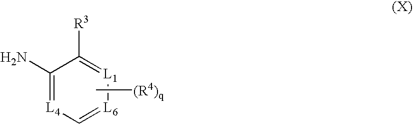

wherein AA is independently selected from halo or triflate; and
Z, R², R³, R⁴, L, p and q are as hereinbefore defined.

In a preferred process of the invention, the product of (a) is reacted in step (b) with a compound of formula (Xb)

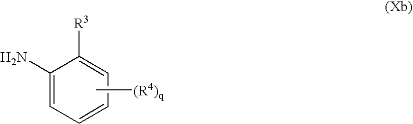

wherein R³, R⁴ and q are as hereinbefore defined.

Preferably, the catalyst used in the processes of the invention is a palladium catalyst.

Preferably AA is a halo group. Preferably still AA is bromide or iodide.

Preferably the coupling reagent is 4-(4,6-Dimethoxy-1,3,5-triazin-1-yl)-4-methyl-morpholinium chloride.

Advantages of this novel process are the minimisation of protection/deprotection steps and the utilisation of a cascade process both of which minimise the number of operations, reaction vessels, waste streams and energy required whilst maximising molecular complexity in a regio- and stereoselective manner.

Furthermore these processes may be carried out on a polymer support which enables large numbers of compounds to be synthesised for screening.

Thus, according to a further aspect of the invention we provide a process for the manufacture of a compound of formula (II) which comprises cleaving a compound of formula (VIII);

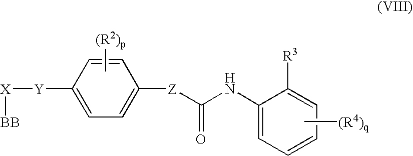

wherein X, Y, Z, R², R³, R⁴, p and q are as hereinbefore defined; and BB is a solid phase resin.

Any conventionally known solid phase resin possessing an —NH— or —NH₂— moeity may be used. However, a preferred resin is Rink Amide MBHA resin.

Compounds of formula (VIII) may be manufactured using the general process;

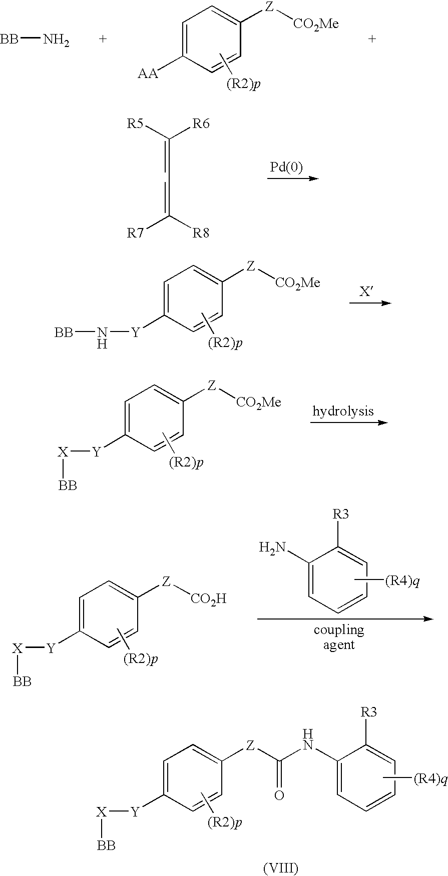

wherein BB, AA, X, Y, Z, R², R³, R⁴, R⁵, R⁶, R⁷, R⁸, p and q are as hereinbefore defined; X′ is the electrophilic precursor of X.

The compounds of the invention are useful, inter alia, as HDAC inhibitors.

Thus, the compounds are suitable for the treatment of a variety of cellular proliferative and/or differentiative disorders including mammalian cancers, for example, haematologic cancers, such as leukaemia, non-small cell lung cancers, colonic cancers, breast cancers, ovarian cancers, renal cancers and melanoma. The compounds of the invention may also be suitable for the treatment of conditions including cystic fibrosis, Huntingdon's chorea and sickle cell anaemia.

Examples of cellular proliferative and/or differentiative disorders include cancer, e.g., carcinoma, sarcoma, metastatic disorders or hematopoietic neoplastic disorders, e.g., leukemias. A metastatic tumor can arise from a multitude of primary tumor types, including but not limited to those of prostate, colon, lung, breast and liver origin.

As used herein, the term “cancer” (also used interchangeably with the terms, “hyperproliferative” and “neoplastic”) refers to cells having the capacity for autonomous growth, i.e., an abnormal state or condition characterized by rapidly proliferating cell growth. Cancerous disease states may be categorized as pathologic, i.e., characterizing or constituting a disease state, e.g., malignant tumor growth, or may be categorized as non-pathologic, i.e., a deviation from normal but not associated with a disease state, e.g., cell proliferation associated with wound repair. The term is meant to include all types of cancerous growths or oncogenic processes, metastatic tissues or malignantly transformed cells, tissues, or organs, irrespective of histopathologic type or stage of invasiveness. The term “cancer” includes malignancies of the various organ systems, such as those affecting lung, breast, thyroid, lymphoid, gastrointestinal, and genito-urinary tract, as well as adenocarcinomas which include malignancies such as most colon cancers, renal-cell carcinoma, prostate cancer and/or testicular tumors, non-small cell carcinoma of the lung, cancer of the small intestine and cancer of the esophagus. The term “carcinoma” is art recognized and refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. Exemplary carcinomas include those forming from tissue of the cervix, lung, prostate, breast, head and neck, colon and ovary. The term “carcinoma” also includes carcinosarcomas, e.g., which include malignant tumors composed of carcinomatous and sarcomatous tissues. An “adenocarcinoma” refers to a carcinoma derived from glandular tissue or in which the tumor cells form recognizable glandular structures. The term “sarcoma” is art recognized and refers to malignant tumors of mesenchymal derivation.

The compounds of the invention can be used to monitor, treat and/or diagnose a variety of proliferative disorders. Such disorders include hematopoietic neoplastic disorders. As used herein, the term “hematopoietic neoplastic disorders” includes diseases involving hyperplastic/neoplastic cells of hematopoietic origin, e.g., arising from myeloid, lymphoid or erythroid lineages, or precursor cells thereof. Preferably, the diseases arise from poorly differentiated acute leukemias, e.g., erythroblastic leukemia and acute megakaryoblastic leukemia. Additional exemplary myeloid disorders include, but are not limited to, acute promyeloid leukemia (APML), acute myelogenous leukemia (AML) and chronic myelogenous leukemia (CML) (reviewed in Vaickus, L. (1991) Crit. Rev. in Oncol./Hemotol. 11:267-97); lymphoid malignancies include, but are not limited to acute lymphoblastic leukemia (ALL) which includes B-lineage ALL and T-lineage ALL, chronic lymphocytic leukemia (CLL), prolymphocytic leukemia (PLL), hairy cell leukemia (HLL) and Waldenstrom's macroglobulinemia (WM). Additional forms of malignant lymphomas include, but are not limited to non-Hodgkin lymphoma and variants thereof, peripheral T cell lymphomas, adult T cell leukemia/lymphoma (ATL), cutaneous T-cell lymphoma (CTCL), large granular lymphocytic leukemia (LGF), Hodgkin's disease and Reed-Sternberg disease.

Examples of cellular proliferative and/or differentiative disorders of the colon include, but are not limited to, non-neoplastic polyps, adenomas, familial syndromes, colorectal carcinogenesis, colorectal carcinoma, and carcinoid tumors.

Examples of cellular proliferative and/or differentiative disorders of the liver include, but are not limited to, nodular hyperplasias, adenomas, and malignant tumors, including primary carcinoma of the liver and metastatic tumors.

Examples of cellular proliferative and/or differentiative disorders of the ovary include, but are not limited to, ovarian tumors such as, tumors of coelomic epithelium, serous tumors, mucinous tumors, endometeriod tumors, clear cell adenocarcinoma, cystadenofibroma, brenner tumor, surface epithelial tumors; germ cell tumors such as mature (benign) teratomas, monodermal teratomas, immature malignant teratomas, dysgerminoma, endodermal sinus tumor, choriocarcinoma; sex cord-stomal tumors such as, granulosa-theca cell tumors, thecoma-fibromas, androblastomas, hill cell tumors, and gonadoblastoma; and metastatic tumors such as Krukenberg tumors.

Examples of cellular proliferative and/or differentiative disorders of the breast include, but are not limited to, proliferative breast disease including, e.g., epithelial hyperplasia, sclerosing adenosis, and small duct papillomas; tumors, e.g., stromal tumors such as fibroadenoma, phyllodes tumor, and sarcomas, and epithelial tumors such as large duct papilloma; carcinoma of the breast including in situ (noninvasive) carcinoma that includes ductal carcinoma in situ (including Paget's disease) and lobular carcinoma in situ, and invasive (infiltrating) carcinoma including, but not limited to, invasive ductal carcinoma, invasive lobular carcinoma, medullary carcinoma, colloid (mucinous) carcinoma, tubular carcinoma, and invasive papillary carcinoma, and miscellaneous malignant neoplasms. Disorders in the male breast include, but are not limited to, gynecomastia and carcinoma.

According to a further aspect of the invention we provide a method of treatment or alleviation of a cellular proliferative and/or differentiative disorder which comprises administering a therapeutically effective amount of compound of formula (II), or a suitable salt thereof as hereinbefore described, to a patient suffering from such a disorder.

In the method of the invention the preferred compounds may be selected from those hereinbefore described.

Thus, according to a yet further aspect of the invention we provide the use of a compound of formula (II) in the manufacture of a medicament for the treatment of a cellular proliferative and/or differentiative disorder.

The method and/or use of the invention preferably treats a cancer selected from leukaemia, colonic cancer, melanoma and non-small cell lung cancer.

More preferably, the cancer is selected from colonic cancer and melanoma, whilst the treatment of colonic cancer is especially preferred.

Features, integers, characteristics, compounds, chemical moieties or groups described in conjunction with a particular aspect, embodiment or example of the invention are to be understood to be applicable to any other aspect, embodiment or example described herein unless incompatible therewith.

The invention will now be described by way of example only.

### EXAMPLES

### General Procedure for the 3-Component Catalytic Cascade

A Schlenk tube was charged with the aryl iodide, the nucleophile (1-3 mol eq), potassium carbonate (2 mol eq), tri-2-furylphosphine (10 mol %), tris (dibenzylideneacetone) dipalladium (0) (2.5 mol %) and acetonitrile followed after two freeze, pump, thaw cycles by allene gas (1 atm, 25° C.). The Schlenk tube was sealed, the mixture was allowed to warm to room temperature and the heated at 80° C. with stirring for 6 to 24 h. The mixture was then cooled, the vessel vented, the mixture concentrated in vacuo and the residue partitioned between dichloromethane and water. The organic layer was separated and the aqueous layer extracted with dichloromethane (3×). The combined organic extracts were dried (MgSO₄), filtered and the filtrate concentrated in vacuo. The residue was purified by flash chromatography and/or crystallisation.

### N-(2-Amino-phenyl)-4-(1-morpholin-4-ylmethyl-vinyl)-benzamide (1)

Prepared by the general procedure using N-(2-amino-phenyl)-4-iodo-benzamide, (170 mg, 0.5 mmol), morpholine (0.045 ml, 1.0 mol eq), potassium carbonate (140 mg, 2.0 mol eq), tri-2-furylphosphine (12 mg, 10 mol %), tris(dibenzylideneacetone) dipalladium (0) (12 mg, 2.5 mol %) and allene gas (1 atm, 25° C.) in acetonitrile (10 ml). The Schlenk tube was heated at 80° C. for 22 h to give (1) which was purified by flash chromatography, eluting with ethyl acetate (R_{F} 0.20) to give colourless plates (153 mg, 91%, m.p 163-164° C.).

Anal: Found: C, 69.5; H, 6.90; N, 11.9. C₂₀H₂₃N₃O₂ 0.5H₂O requires: C, 69.3; H, 6.69; N, 12.1%.

δ ¹H (300 MHz): 7.87 (d, 2H, J 8.4 Hz, ArH), 7.66 (d, 2H, J 8.4 Hz, ArH), 7.34 (d, 1H, J 7.9 Hz, ArH), 7.14-7.08 (m, 1H, ArH), 6.89-6.84 (m, 2H, ArH), 5.60 (d, 1H, ²J 0.9 Hz, C═CH), 5.35 (d, 1H, ²J 0.8 Hz, C═CH), 3.87 (br s, 2H, NH₂), 3.67 (t, 4H, J 4.6 Hz, OCH₂), 3.37 (s, 4H, C═CCH₂), 2.47 (t, 4H, J 4.4 Hz, CH₂CH₂N). δ ¹³C (75 MHz): 175.8 (C═O), 144.1 (C═C), 143.2, 141.1, 133.4, 127.6, 127.1, 125.6, 125.0, 120.2, 118.8, 117.9 (C═C), 67.4 (C—O), 63.8 (C—N), 53.9 (C—N).

m/z (CI+, %): 338.3 (M+H, 29).

HRMS: Found [M+H] 338.1866. C₂₀H₂₃N₃O₂ requires 338.1868.

IR (vₘₐₓ/cm⁻¹): 3423 (CONH), 3340 (NH₂), 3293 (CONH), 1641 (CONH), 1611 (C═C)

### N-(2-amino-phenyl)-4-(1-{[benzyl(methyl)amino]methyl}vinyl)benzamide (2)

Prepared by the general procedure using N-(2-Amino-phenyl)-4-iodo-benzamide, (170 mg, 0.5 mmol), N-benzylmethylamine (0.071 ml, 1.1 mol eq), potassium carbonate (140 mg, 2.0 mol eq), tri-2-furylphosphine (12 mg, 10 mol %), tris (dibenzylideneacetone) dipalladium (0) (12 mg, 2.5 mol %) and allene gas (1 atm, 25° C.) in acetonitrile (10 ml). The Schlenk tube was heated at 80° C. for 22 h to give (2) which was purified by gradient flash chromatography, eluting with ether-hexane (1:1 (v/v)), (2:1 (v/v)), (3:1 (v/v) thereafter) (R_{F} 0.24) to give colourless prisms (175 mg, 94%, m.p 110-111° C.).

Anal: Found: C, 75.5; H, 6.70; N, 10.9. C₂₄H₂₅N₃O 0.5H₂O requires: C, 75.8; H, 6.75; N, 11.0%.

δ ¹H (300 MHz): 7.97 (s, 1H, ArH), 7.83 (d, 2H, J 8.3 Hz, ArH), 7.53 (d, 2H, J 8.4 Hz, ArH), 7.33-7.23 (m, 5H, ArH), 7.11-7.05 (m, 1H, ArH), 6.85-6.80 (m, 2H, ArH), 5.55 (d, 1H, ²J 0.8 Hz, C═CH), 5.38 (s, 1H, C═CH), 3.87 (br s, 2H, NH₂), 3.52 (s, 2H, PhCH₂), 3.39 (s, 2H, C═CCH₂), 2.18 (s, 3H, NMe). δ ¹³C (75 MHz): 166.1 (C═O), 145.0 (C═C), 144.0, 141.2, 139.3, 133.3, 129.5, 128.7, 127.6, 127.5, 127.3, 125.7, 125.0, 120.2, 118.8, 117.6 (C═C), 62.4 (C—N), 62.2 (C—N), 42.5 (C—N).

m/z (ES+, %): 372.2 (M+H, 64).

IR (vₘₐₓ/cm⁻¹): 3418 (CONH), 3346 (NH₂), 3294 (CONH), 1636 (CONH), 1602 (C═C)

### N-(2-Amino-phenyl)-4-(1-{[(pyridin-3-ylmethyl)amino]methyl}vinyl)benzamide (3)

Prepared by the general procedure using N-(2-amino-phenyl)-4-iodo-benzamide, (187 mg, 0.5 mmol), 3-(aminomethyl)pyridine (0.051 ml, 1.0 mol eq), potassium carbonate (140 mg, 2.0 mol eq), tri-2-furylphosphine (12 mg, 10 mol %), tris (dibenzylideneacetone) dipalladium (0) (12 mg, 2.5 mol %) and allene gas (1 atm, 25° C.) in acetonitrile (10 ml). The Schlenk tube was heated at 80° C. for 21 h to give (3) which was purified by gradient flash chromatography, eluting with ethyl acetate-methanol (19:1 (v/v)), (9:1 (v/v) thereafter) (R_{F} 0.05) to give a yellow oil (129 mg, 72%).

δ ¹H (300 MHz): 8.53-8.46 (m, 2H, ArH), 8.32 (s, 1H, ArH), 7.86 (d, 2H, J 8.2 Hz, ArH), 7.64 (d, 1H, J 7.8 Hz, ArH), 7.48 (d, 2H, J 8.2 Hz, ArH), 7.29-7.21 (m, 1H, ArH), 7.09-7.04 (m, 1H, ArH), 6.81 (d, 2H, J 7.7 Hz, ArH), 5.52 (s, 1H, C═CH), 5.35 (s, 1H, C═CH), 3.79 (s, 2H, NCH₂), 3.68 (s, 2H, NCH₂), 3.15 (br s, 2H, NH₂).

δ ¹³C (CDCl₃, 75 MHz): 166.0 (C═O), 150.1, 148.9, 145.5, 143.5, 141.3, 136.5, 135.7, 133.7, 128.0, 127.6, 126.9, 125.8, 124.9, 123.9, 120.0, 118.7, 116.3 (C═C), 53.0 (C—N), 50.6 (C—N).

m/z (ES+, %): 359.0 (M+H, 82).

HRMS: Found [M+H] 359.1866. C₂₂H₂₂N₄O requires 359.1866.

IR (vₘₐₓ/cm⁻¹): 3368 (CONH), 3274 (CONH), 1649 (CONH), 1610 (C═C)

### N-(2-Amino-phenyl)-4-[1-(3,4-dihydroisoquinolin-2(1H)-ylmethyl)vinyl]benzamide (4)

Prepared by the general procedure using N-(2-amino-phenyl)-4-iodo-benzamide, (170 mg, 0.5 mmol), tetrahydroisoquinoline (0.070 ml, 1.1 mol eq), potassium carbonate (140 mg, 2.0 mol eq), tri-2-furylphosphine (12 mg, 10 mol %), tris (dibenzylideneacetone) dipalladium (0) (12 mg, 2.5 mol %) and allene gas (1 atm, 25° C.) in acetonitrile (10 ml). The Schlenk tube was heated at 80° C. for 24 h to give (4) which was purified by flash chromatography, eluting with 1.5:1 (v/v) ether-hexane (R_{F} 0.10) and thereafter by crystallisation from dichloromethane to give colourless needles (165 mg, 86%, m.p 146° C.)

Anal: Found: C, 77.9; H, 6.60; N, 10.9. C₂₅H₂₅N₃O requires: C, 78.2; H, 6.57; N, 11.0%.

δ ¹H (300 MHz): 7.85 (d, 2H, J 8.4 Hz, ArH), 7.69 (d, 2H, J 8.4 Hz, ArH), 7.32 (d, 1H, J 7.7 Hz, ArH), 7.13-7.00 (m, 5H, ArH), 6.99-6.82 (m, 2H, ArH), 5.63 (d, 1H, ²J 1.2 Hz, C═CH), 5.42 (d, 1H, ²J 1.0 Hz, C═CH), 3.85 (br s, 2H, NH₂), 3.67 (s, 2H, NCH₂), 3.55 (s, 2H, C═CCH₂), 2.88-2.76 (m, 4H, NCH₂CH₂).

δ ¹³C (75 MHz): 166.0 (C═O), 144.2 (C═C), 143.9, 141.0, 135.3, 134.9, 129.1, 127.6, 127.2, 127.0, 126.5, 126.4, 126.0, 125.5, 125.0, 120.2, 118.8, 117.6 (C═C), 63.1 (C—N), 56.3 (C—N), 50.8 (C—N), 29.5 (C—C).

m/z (ES+, %): 384.1 (M+H, 54).

IR (vₘₐₓ/cm⁻¹): 3455 (CONH), 3340 (NH₂), 3245 (CONH), 1624 (CONH), 1597 (C═C)

### N-(2-Amino-phenyl)-4-{1-[(4-pyridin-2-ylpiperazin-1-yl)methyl]vinyl}benzamide (5)

Prepared by the general procedure using N-(2-amino-phenyl)-4-iodo-benzamide, (170 mg, 0.5 mmol), 1-(2-pyridyl)piperazine (0.084 ml, 1.1 mol eq), potassium carbonate (140 mg, 2.0 mol eq), tri-2-furylphosphine (12 mg, 10 mol %), tris (dibenzylideneacetone) dipalladium (0) (12 mg, 2.5 mol %) and allene gas (1 atm, 25° C.) in acetonitrile (10 ml). The Schlenk tube was heated at 80° C. for 22 h to give (5) which was purified by flash chromatography, eluting with 19:1 (v/v) ether-methanol (R_{F} 0.01) and thereafter by crystallisation from dichloromethane/hexane to give colourless prisms (161 mg, 78%, m.p 111-112° C.)

Anal: Found: C, 72.4; H, 6.55; N, 16.6. C₂₅H₂₇N₅O requires: C, 72.6; H, 6.58; N, 16.9%.

δ ¹H (300 MHz): 8.18 (dd, 1H, J 4.8 Hz, ⁴J 1.4 Hz, ArH), 8.09 (s, 1H, ArH), 7.84 (d, 2H, J 8.2 Hz, ArH), 7.63 (d, 2H, J 8.3 Hz, ArH), 7.48-7.38 (m, 1H, ArH), 7.27 (d, 1H, J 9.2 Hz, ArH), 7.06 (d, 1H, J 7.6 Hz, ArH), 6.82-6.78 (m, H, ArH), 6.63-6.53 (m, 2H, ArH), 5.60 (s, 1H, C═CH), 5.36 (s, 1H, C═CH), 3.49 (t, 4H, J 4.7 Hz, NCH₂), 3.40 (s, 2H, CH₂C═C), 2.57 (t, 4H, NCH₂).

δ ¹³C (75 MHz): 160.0 (C═O), 148.3, 144.2, 143.5, 141.2, 137.9, 133.4, 127.7, 127.6, 127.1, 125.6, 125.0, 123.9, 120.2, 118.8, 117.8, 113.7 (C═C), 107.5, 63.4 (C—N), 53.2 (C—N), 45.6 (C—N).

m/z (ES+, %): 414.1 (M+H, 22).

IR (vₘₐₓ/cm⁻¹): 3302 (CONH), 1649 (CONH), 1621 (C═C) N-(2-aminophenyl)-4-[1-(1,3,4,9-tetrahydro-2H-β-carbolin-2-ylmethyl)vinyl]benzamide (6)

Prepared by the general procedure using N-(2-amino-phenyl)-4-iodo-benzamide, (200 mg, 0.6 mmol), 1,2,3,4-tetrahydro-9H-pyrido[3,4-b]indole (114 mg, 1.1 mol eq), potassium carbonate (168 mg, 2.0 mol eq), tri-2-furylphosphine (14 mg, 10 mol %), tris(dibenzylideneacetone) dipalladium (0) (14 mg, 2.5 mol %) and allene gas (1 atm, 25° C.) in acetonitrile (10 ml). The Schlenk tube was heated at 80° C. for 6 h to give (6) which was purified by flash chromatography, eluting with 1:1 (v/v) ether-hexane (R_{F} 0.10) and thereafter by crystallisation from dichloromethane/hexane to give pale yellow prisms (145 mg, 57%, m.p 172-173° C.).

Anal: Found: C, 75.3; H, 6.25; N, 12.8. C₂₇H₂₆N₄O 0.5H₂O requires: C, 75.1; H, 6.19; N, 13.0%.

δ ¹H (500 MHz): 7.85 (d, 2H, J 8.2 Hz, ArH), 7.80 (s, 1H, ArH), 7.70 (d, 2H, J 8.4 Hz, ArH), 7.46 (d, 1H, J 7.2 Hz, ArH), 7.33-7.26 (m, 1H, ArH), 7.18-7.05 (m, 3H, ArH), 6.86-6.82 (m, 2H, ArH), 5.63 (s, 1H, C═CH), 5.42 (s, 1H, C═CH), 3.72 (s, 2H, NCH₂), 3.66 (s, 2H, NCH₂), 2.95 (t, 2H, J 5.7 Hz, NCH₂CH₂), 2.79 (t, 2H, J 5.5 Hz, CH₂CH₂N).

δ ¹³C (75 MHz): 165.5 (C═O), 144.3 (C═C), 143.4, 142.6, 136.1, 133.9, 133.1, 128.0, 126.9, 126.8, 126.6, 123.6, 120.1, 118.6, 117.7, 116.6, 116.4, 111.2, 106.8 (C═C), 62.0 (C—N), 50.7 (C—N), 50.0 (C—N), 21.4 (C—C).

m/z (ES+, %): 423.0 (M+H, 73).

HRMS: Found [M+H] 423.2183. C₂₇H₂₆N₄O requires 423.2179.

IR (vₘₐₓ/cm⁻¹): 3322 (NH₂), 3172 (CONH), 1657 (CONH), 1623 (C═C)

### N-(2-aminophenyl)-4-{1-[(benzylamino)methyl]vinyl}benzamide (7)

Prepared by the general procedure using N-(2-amino-phenyl)-4-iodo-benzamide, (190 mg, 0.56 mmol), benzylamine (0.184 ml, 3 mol eq), potassium carbonate (155 mg, 2 mol eq), tri-2-furylphosphine (13 mg, 10 mol %), tris(dibenzylideneacetone) dipalladium (0) (13 mg, 2.5 mol %) and allene gas (1 atm, 25° C.) in acetonitrile (10 ml). The Schlenk tube was heated at 80° C. for 21.5 h to give (7) which was purified by flashchromatography, eluting with 19:1 (v/v) ether-methanol (R_{F} 0.08) to give a yellow amorphous solid (103 mg, 52%, m.p 89-91° C.).

Anal: Found: C, 75.4; H, 6.45; N, 11.5. C₂₃H₂₃N₃O.0.5H₂O requires: C, 75.4; H, 6.46; N, 11.5%.

δ ¹H (300 MHz): 7.98 (br s, 1H, ArH), 7.90 (d, 2H, J 8.0 Hz, ArH), 7.52 (d, 2H, J 8.0 Hz, ArH), 7.30-7.19 (m, 4H, ArH), 7.10 (t, 2H, J 5.0 Hz, ArH), 6.90-6.80 (m, 2H, ArH), 5.53 (s, 1H, C═CH), 5.37 (s, 1H, C═CH), 3.81 (s, 2H, PhCH₂N), 3.69 (s, 2H, NCH₂C═C), 1.41 (s, 1H, NH).

δ ¹³C (75 MHz): 165.9 (C═O), 145.8 (C═C), 143.9, 141.1, 140.4, 133.5, 128.9, 128.6, 127.9, 127.6, 127.5, 127.0, 125.6, 120.2, 118.8, 115.8 (C═C), 53.5 (C—N), 53.0 (C—N)

m/z (ES+, %): 358.1 (M+H, 48).

IR (vₘₐₓ/cm⁻¹): 3456 (CONH), 3351 (NH), 1648 (CONH), 1624 (C═C)

### N-(2-aminophenyl)-4-{1-({4-3-(trifluoromethylphenyl]piperazin1yl}methyl)vinyl]benzamide (8)

Prepared by the general procedure using N-(2-amino-phenyl)-4-iodo-benzamide, (200 mg, 0.59 mmol), 1-(α,α,α-trifluoro-m-tolyl)piperazine (0.122 ml, 1.1 mol eq), potassium carbonate (163 mg, 2.0 mol eq), tri-2-furylphosphine (14 mg, 10 mol %), tris(dibenzylideneacetone) dipalladium (0) (14 mg, 2.5 mol %) and allene gas (1 atm, 25° C.) in acetonitrile (10 ml). The Schlenk tube was heated at 80° C. for 22.5 h to give (8) which was purified by gradient flash chromatography, eluting with ether (800 ml) and thereafter 19:1 (v/v) ether-methanol (R_{F} 0.05) to give colourless plates (236 mg, 83%, m.p 94-96° C.).

Anal: Found: C, 67.5; H, 5.66; N, 11.7; F, 11.9. C₂₇H₂₇N₄O requires: C, 67.4; H, 5.65; N, 11.7; F, 11.9%.

δ ¹H (500 MHz): 7.88 (d, 2H, J 8.1 Hz, ArH), 7.68 (d, 2H, J 8.2 Hz, ArH), 7.35-7.31 (m, 2H, ArH), 7.10-7.04 (m, 4H, ArH), 6.87-6.80 (m, 2H, ArH), 5.62 (s, 1H, C═CH), 5.39 (s, 1H, C═CH), 3.87 (br s, 2H, NH₂), 3.44 (s, 2H, NCH₂C═C), 3.21 (t, 4H, J 5.1 Hz, NCH₂CH₂N), 2.65 (t, 4H, J 5.0 Hz, NCH₂CH₂N).

δ ¹³C (75 MHz): 165.5 (C═O), 151.8, 144.1 (C═C), 143.5, 141.1, 133.4, 131.8 (C—F), 129.9, 127.9, 127.7, 127.1, 125.6, 125.0, 120.2, 119.1, 118.8, 117.9, 116.1, 112.5 (C═C), 63.3 (C—N), 53.2 (C—N), 49.1 (C—N).

m/z (ES+, %): 481.1 (M+H, 50).

IR (vₘₐₓ/cm⁻¹): 3423 (CONH), 1631 (CONH), 1607 (C═C).

### N-(2-aminophenyl)-4-{1-[4-(2-methoxy-phenyl)-piperazin-1-ylmethyl]vinyl}-benzamide (9)

Prepared by the general procedure using N-(2-amino-phenyl)-4-iodo-benzamide, (170 mg, 0.5 mmol), 1-(2-methoxyphenyl)piperazine (106 mg, 1.1 mol eq), potassium carbonate (140 mg, 2 mol eq), tri-2-furylphosphine (12 mg, 10 mol %), tris (dibenzylideneacetone) dipalladium (0) (12 mg, 2.5 mol %) and allene gas (1 atm, 25° C.) in acetonitrile (10 ml). The Schlenk tube was heated at 80° C. for 24 h to give (9) which was purified by flash chromatography, eluting with ether (R_{F} 0.04) to give colourless plates (134 mg, 61%, m.p 115-116° C.).

Anal: Found: C, 72.9; H, 6.75; N, 12.9. C₂₇H₃₀N₄O₂ requires: C, 73.2; H, 6.83; N, 12.7%. δ ¹H (500 MHz): 7.89 (d, 2H, J 8.1 Hz, ArH), 7.80 (br s, 1H, ArH), 7.69 (d, 2H, J 8.2 Hz, ArH), 7.34 (d, 1H, J 7.0 Hz, ArH), 7.10 (t, 1H, J 5.0 Hz, ArH), 6.99-6.91 (m, 1H, ArH), 6.88-6.80 (m, 4H, ArH), 5.61 (s, 1H, C═CH), 5.38 (s, 1H, C═CH), 3.88 (br s, 2H, NH₂), 3.86 (s, 3H, OMe), 3.44 (s, 2H, NCH₂C═C), 3.05 (br s, 4H, NCH₂CH₂N), 2.67 (br s, 4H, J 5.0 Hz, NCH₂CH₂N).

δ ¹³C (75 MHz): 169.5 (C═O), 152.7, 144.4 (C═C), 143.7, 141.8, 133.3, 127.6, 127.2, 125.5, 125.1, 123.2, 121.3, 120.2, 118.9, 117.7, 111.5 (C═C), 63.5 (C—O), 55.7 (C—N), 53.7 (C—N), 51.1 (C—N)

m/z (ES+, %): 443.0 (M+H, 33).

HRMS: Found [M+H] 443.2437. C₂₇H₃₀N₄O₂ requires 443.2442.

IR (vₘₐₓ/cm⁻¹): 3456 (CONH), 1634 (CONH), 1606 (C═C).

### N-(2-aminophenyl)-4-(1-[4-(4-fluoro-phenyl)-piperazin-1-ylmethyl]-vinyl)-benzamide (10)

Prepared by the general procedure using N-(2-amino-phenyl)-4-iodo-benzamide, (170 mg, 0.5 mmol), 1-(4-fluorophenyl)piperazine (106 mg, 1.1 mmol eq), potassium carbonate (140 mg, 2 mol eq), tri-2-furylphosphine (12 mg, 10 mol %), tris (dibenzylideneacetone) dipalladium (0) (12 mg, 2.5 mol %) and allene gas (1 atm, 25° C.) in acetonitrile (10 ml). The Schlenk tube was heated at 80° C. for 24 h to give (10) which was purified by flash chromatography, eluting with ether (R_{F} 0.04) to give colourless plates (134 mg, 61%, m.p 131-132° C.).

Anal: Found: C, 71.5; H, 6.30; N, 13.0; F, 4.2. C₂₆H₂₇FN₄0.0.25H₂O requires: C, 71.8; H, 6.26; N, 12.9; F, 4.4%.

δ ¹H (300 MHz): 7.87 (d, 2H, J 8.3 Hz, ArH), 7.68 (d, 2H, J 8.4 Hz, ArH), 7.33 (d, 1H, J 7.6 Hz, ArH), 7.10 (dt, H, J 7.7 Hz, ⁴J 1.5 Hz, ArH), 6.93 (d, 2H, J 8.3 Hz, ArH), 6.92-6.84 (m, 4H, ArH), 5.62 (d, 1H, ²J 0.9 Hz, C═CH), 5.38 (s, 1H, C═CH), 3.88 (br s, 2H, NH₂), 3.44 (s, 2H, NCH₂C═C), 3.09 (t, 4H, J 4.8 Hz, NCH₂CH₂N), 2.64 (t, 4H, J 5.0 Hz, NCH₂CH₂N).

δ ¹³C (75 MHz): 159.1 (C═O), 156.0 (C—F), 148.4 (C═C), 144.2, 143.6, 141.0, 133.4, 127.8, 127.6, 127.2, 126.8, 125.5, 125.0, 120.2, 118.9, 118.1, 117.8, 116.0, 115.7 (C═C), 63.3 (C—N), 53.4 (C—N), 50.6 (C—N)

m/z (ES+, %): 431.1 (M+H, 20).

HRMS: Found [M+H] 431.2241. C₂₆H₂₇FN₄O requires 431.2242.

IR (vₘₐₓ/cm⁻¹): 3428 (CONH), 1640 (CONH), 1604 (C═C).

### N-(2-Amino-phenyl)-2-[4-(1-morpholin-4-ylmethyl-vinyl)-phenyl]-acetamide (11)

Prepared by the general procedure using N-(2-amino-phenyl)-2-(4-iodo-phenyl)-acetamide (200 mg, 0.57 mmol), morpholine (50 μl, 1.0 mol eq), potassium carbonate (158 mg, 2.0 mol eq), tri-2-furylphosphine (13 mg, 10 mol %), tris (dibenzylideneacetone) dipalladium (0) (13 mg, 2.5 mol %) and allene gas (1 atm, 25° C.) in acetonitrile (10 ml). The Schlenk tube was heated at 80° C. for 24 h to give (11) which was purified by flash chromatography, eluting with 9:1 (v/v) ethyl acetate-hexane (R_{F} 0.12) to give a colourless amorphous solid (140 mg, 70%, m.p 129° C.).

Anal: Found: C, 71.6; H, 7.05; N, 12.0. C₂₁H₂₅N₃O₂ requires: C, 71.8; H, 7.17; N, 12.0%.

δ ¹H (500 MHz): 7.58 (d, 2H, J 7.8 Hz, ArH), 7.32 (d, 2H, J 7.8 Hz, ArH), 7.10 (d, 1H, J 7.9 Hz, ArH), 7.05-7.00 (m, 2H, ArH), 6.76-6.75 (m, 2H, ArH and NH), 5.52 (s, 1H, C═CH), 5.27 (s, 1H, C═CH), 3.78 (s, 2H, ArCH₂), 3.72 (br s, 2H, NH₂), 3.68 (t, 4H, J 4.0 Hz, OCH₂), 3.33 (s, 2H, NCH₂), (t, 4H, J 4.1 Hz, OCH₂CH₂N).

δ ¹³C (75 MHz): 169.7 (C═O), 143.0 (C═C), 140.7, 139.6, 133.7, 129.3, 127.4, 127.2, 125.1, 123.9, 119.5, 116.1 (C═C), 67.1 (C—O), 63.5 (C—N), 53.5 (C—N), 43.9 (C—C).

m/z (ES+, %): 352.1 (M+H, 55), 197.0 (86), 176.5 (100).

IR (vₘₐₓ/cm⁻¹): 3393 and 3310 (CONH), 1644 (CONH), 1610 (C═C)

### N-(2-Amino-phenyl)-3-[4-(J-morpholin-4-ylmethyl-vinyl)-phenyl]-propionamide (12)

Prepared by the general procedure using N-(2-amino-phenyl)-3-(4-iodo-phenyl)-propionamide (125 mg, 0.34 mmol), morpholine (30 μl, 1.0 mol eq), potassium carbonate (94 mg, 2.0 mol eq), tri-2-furylphosphine (8 mg, 10 mol %), tris (dibenzylideneacetone) dipalladium (0) (8 mg, 2.5 mol %) and allene gas (1 atm, 25° C.) in acetonitrile (10 ml). The Schlenk tube was heated at 80° C. for 23 h to give (12) which was purified by flash chromatography, eluting with 9:1 (v/v) ethyl acetate-hexane (R_{F} 0.11) to give colourless needles (93 mg, 75%, m.p 103° C.).

Anal: Found: C, 72.1; H, 7.45; N, 11.6. C₂₂H₂₇N₃O₂ requires: C, 72.3; H, 7.45; N, 11.5%.

δ ¹H (300 MHz): 7.49 (d, 2H, J 8.1 Hz, ArH), 7.23 (d, 2H, J 8.1 Hz, ArH), 7.08 (d, 1H, J 7.6 Hz, ArH), 7.02 (d, 1H, J 7.6 Hz, ArH), 6.84 (br s, 1H, NH), 6.75 (t, 2H, J 7.5 Hz, ArH), 5.47 (s, 1H, C═CH), 5.23 (s, 1H, C═CH), 3.68 (t, 4H, J 4.6 Hz, OCH₂), 3.54 (s, 2H, NH₂), 3.32 (s, 2H, NCH₂C═C), 3.07 (t, 2H, J 7.3 Hz, ArCH₂)), 2.71 (t, 2H, J 7.3 Hz, ArCH₂CH₂), 2.47 (t, 4H, J 4.6 Hz, OCH₂CH₂N).

δ ¹³C (75 MHz): 165.9 (C═O), 138.4 (C═C), 136.0, 135.0, 133.6, 123.5, 122.4, 121.8, 120.6, 119.2, 114.5, 112.9, 110.8 (C═C), 62.2 (C—O), 58.8 (C—N), 48.7 (C—N), 34.0 (C—C), 26.7 (C—C).

m/z (ES+, %): 366.4 (M+H, 42), 204.2 (56), 183.8 (100).

IR (vₘₐₓ/cm⁻¹): 3434 and 3363 (CONH), 1651 (CONH), 1615 (C═C)

### N-(2-Amino-phenyl)-4-[4-(1-morpholin-4-ylmethyl-vinyl)-phenyl]-butanamide (13)

Prepared by the general procedure using N-(2-amino-phenyl)-4-(4-iodo-phenyl)-butyramide (220 mg, 0.58 mmol), morpholine (58 μl, 1.0 mol eq), potassium carbonate (160 mg, 2.0 mol eq), tri-2-furylphosphine (13 mg, 10 mol %), tris (dibenzylideneacetone) dipalladium (0) (13 mg, 2.5 mol %) and allene gas (1 atm, 25° C.) in acetonitrile (10 ml). The Schlenk tube was heated at 80° C. for 23 h to give (13) which was purified by flash chromatography, eluting with 9:1 (v/v) ethyl acetate-hexane (R_{F} 0.16) to give colourless plates (166 mg, 75%, m.p 79-81° C.).

Anal: Found: C, 72.8; H, 7.75; N, 11.1. C₂₃H₂₉N₃O₂ requires: C, 72.8; H, 7.70; N, 11.1%.

δ ¹H (300 MHz): 7.48 (d, 2H, J 8.0 Hz, ArH), 7.17 (d, 2H, J 7.9 Hz, ArH), 7.15 (br s, 1H, NH), 7.06 (m, 2H, ArH), 6.80 (d, 2H, J 6.3 Hz, ArH), 5.48 (s, 1H, C═CH), 5.22 (s, 1H, C═CH), 3.68 (t, 4H, J 4.4 Hz, OCH₂), 3.31 (s, 2H, NCH₂C═C), 2.73 (t, 2H, J 7.4 Hz, ArCH₂), 2.48 (t, 4H, J 4.3 Hz, OCH₂CH₂N), 2.41 (t, 2H, 7.4 Hz, ArCH₂CH₂CH₂), 2.09 (quintet, 2H, J 7.5 Hz, ArCH₂CH₂).

δ ¹³C (75 MHz): 171.3 (C═O), 143.2 (C═C), 140.8, 140.7, 128.4, 127.2, 126.4, 125.1, 124.3, 119.6, 118.3, 115.2 (C═C), 67.1 (C—O), 63.6 (C—N), 53.6 (C—N), 36.1 (C—C), 34.8 (C—C), 27.0 (C—C).

m/z (ES+, %): 380.4 (M+H, 36), 211.2 (28), 190.7 (100).

IR (vₘₐₓ/cm⁻¹): 3433 and 3363 (CONH), 1650 (CONH), 1610 (C═C)

### N-(2-Amino-phenyl)-4-[1-(3,4-dihydro-1H-isoquinolin-2-ylmethyl)-2-methyl-propenyl]-benzamide (14)

A Schlenk tube was charged with N-(2-amino-phenyl)-4-iodo-benzamide (200 mg, 0.59 mmol), 1,2,3,4-tetrahydroisoquinoline (74 μl, 1.0 mol eq), 3-methyl-1,2-butadiene (70 μl, 1.2 mol eq), potassium carbonate (163 mg, 2.0 mol eq), tri-2-furylphosphine (14 mg, 10 mol %), tris(dibenzylideneacetone) dipalladium (0) (14 mg, 2.5 mol %) and acetonitrile (10 ml) followed after one freeze, pump, thaw cycle by nitrogen. The mixture was allowed to warm to room temperature and then heated at 80° C. with stirring for 24 h. The mixture was then cooled, vented, concentrated in vacuo and the residue partitioned between dichloromethane (20 ml) and water (20 ml). The organic layer was separated and the aqueous layer extracted with dichloromethane (3×10 ml). The combined organic extracts were dried (MgSO₄), filtered and concentrated in vacuo to give (14) which was purified by flash chromatography, eluting with ether (R_{F} 0.09) to give colourless plates (164 mg, 69%, m.p 120° C.).

Anal: Found: C, 78.7; H, 7.15; N, 10.1. C₂₇H₂₉N₃O requires: C, 78.8; H, 7.10; N, 10.2%.

δ ¹H (500 MHz): 7.86 (br s, 1H, NH), 7.79 (d, 2H, J 8.2 Hz, ArH), 7.32-7.29 (m, 3H, ArH), 7.10-7.04 (m, 4H, ArH), 6.99-6.96 (m, 1H, ArH), 6.83-6.79 (m, 2H, ArH), 3.80 (br s, 2H, NH₂), 3.59 (s, 2H, ArCH₂N), 3.42 (s, 2H, NCH₂C═C), 2.79 (t, 2H, J 5.5 Hz, ArCH₂CH₂N), 2.70 (t, 2H, J 5.4 Hz, ArCH₂CH₂N), 1.94 (s, 3H, CH₃), 1.64 (s, 3H, CH₃).

δ ¹³C (75 MHz): 166.0 (C═O), 147.8 (C═C), 140.7, 135.2, 134.7, 133.7, 131.7, 131.2, 129.5, 128.6, 127.1, 126.9, 126.5, 125.9, 125.5, 125.2, 124.6, 119.7, 118.3 (C═C), 65.9 (C—N), 60.0 (C—N), 50.0 (C—N), 29.2 (Ar—C), 22.7 (CH₃), 20.7 (CH₃).

m/z (ES+, %): 412.3 (M+H, 44), 227.2 (100).

IR (vₘₐₓ/cm⁻¹): 1654 (C═O).

### General Procedure for the Solid-Phase “Catch and Release” 3-Component Catalytic Cascade.

### Step A. Removal of the Fmoc Protecting Group

To a 50 ml RB flask containing Rink Amide MBHA Resin (loading 0.73 mmol/g) was added 10 ml of 20% (v/v) piperidine in DMF. The solution was agitated at room temperature for 1 h, then filtered and washed with DMF (3×10 ml) and DCM (10 ml). The resin was dried in vacuo and used directly in the next step.

### Step B. 3-Component Catalytic Cascade

A Schlenk tube was charged with the resin from Step A, the aryl iodide (1.1 mol eq), potassium carbonate (2.0 mol eq), tri-2-furylphosphine (10 mol %), tris (dibenzylideneacetone) dipalladium (0) (2.5 mol %) and DMF, followed after two freeze, pump, thaw cycles by allene gas (1 atm, 25° C.). The Schlenk tube was sealed and the mixture was allowed to warm to room temperature and then heated at 80-100° C. with gentle stirring for 16 to 24 h. The mixture was then cooled, the Schlenk tube vented, and the mixture filtered and washed with DCM, H₂O, MeOH and DCM. The resin was dried in vacuo and used directly in the next step.

### Step C. Acylation of the Cascade Product

The acylating agent (2.0 mol eq) in anhydrous DCM was added dropwise to a stirred mixture of the resin from Step B, triethylamine (3.0 mol eq) and anhydrous DCM under nitrogen at 0° C. The reaction mixture was allowed to warm to room temperature and was agitated for 16 to 24 h then filtered and washed with DCM, H₂O, MeOH and DCM. The resin was dried in vacuo and used directly in the next step.

### Step D: Silanolate Conversion of Ester to Acid

The resin from step C was added to an agitated slurry of potassium trimethyl silanolate (4.0 mol eq) in anhydrous DCM at room temperature under nitrogen. The reaction mixture was agitated for 16 h and the resin filtered and washed with MeOH, water and MeOH. The resin was then acidified with dilute acetic acid in THF (1:2), filtered again and washed with MeOH, water, MeOH and DCM. The resin was dried in vacuo and used directly in the next step.

Step E: Coupling of Acid-Resin with Aniline

The resin from step D and 1,2-phenylenediamine (3.0 mol eq) in dry DMF were agitated at room temperature for 10 min. 4-(4,6-Dimethoxy-1,3,5-triazin-2-yl)-4-methylmorpholinium chloride (DMTMM) (2.0 mol eq) was added and the mixture agitated for a further 16 h. The resin was filtered and washed with MeOH, water, MeOH and DCM. The resin was dried in vacuo and used directly in the next step.

Step F: Cleavage from the Resin to Give Product

The resin from step E was slurried in 20% (v/v) trifluoroacetic acid (TFA) in DCM, the resulting mixture was allowed to stand at room temperature for 20 min then filtered and washed with DCM. The combined filtrates were evaporated under reduced pressure to yield the product which was immediately analysed for purity by HPLC (recorded at 254 nm using a Luna 5μ (250×4.6 mm) phenyl-hexyl column) and was, if necessary, further purified by flash chromatography and/or crystallisation.

### N-(2-Amino-phenyl)-4-[1-(benzoylamino-methyl)-vinyl]-benzamide (18)

The general procedure was followed using:

Step A: Rink Amide MBHA Resin (0.68 g, 0.50 mmol) agitated in 10 ml of 20% (v/v) piperidine in DMF for 1 h to give 18a.
Step B: Resin 18a (0.50 mmol), methyl 4-iodobenzoate (167 mg, 1.1 mol eq), potassium carbonate (138 mg, 2.0 mol eq), tri-2-furylphosphine (12 mg, 10 mol %), tris(dibenzylideneacetone) dipalladium (0) (12 mg, 2.5 mol %) and allene gas (1 atm, 25° C.) in DMF (10 ml). The Schlenk tube was heated at 80° C. for 26 h to give 18b.
Step C: Benzoyl chloride (0.174 ml, 3.0 mol eq) in DCM (2 ml) was added dropwise to a gently stirred solution of 18b (0.50 mmol) and triethylamine (0.266 ml, 4.0 mol eq) in DCM (10 ml) at 0° C. Mixture was agitated at room temperature for 18 h to give 18c.
Step D: Resin 18c was added to an agitated slurry of potassium trimethyl silanolate (257 mg, 4.0 mol eq) in dry DCM (10 ml) at room temperature. The mixture was agitated for 16 h to give 18d.
Step E: Resin 18d and 1,2-phenylenediamine (162 mg, 1.5 mmol) in dry DMF (10 ml) were agitated at room temperature for 10 min. 4-(4,6-Dimethoxy-1,3,5-triazin-2-yl)-4-methylmorpholinium chloride (DMTMM) (277 mg, 1.0 mmol) was added and the mixture agitated for a further 16 h to give 18e.
Step F: Resin 18e was slurried in 20% (v/v) TFA in DCM (10 ml) and allowed to stand for 20 min to give (18) (purity 94% by HPLC) as a colourless solid (118 mg, 64% overall yield from Step A).

δ ¹H (300 MHz, DMSO-_{d6}): 9.70 (s, 1H, NH), 8.86 (t, 1H, J 5.7 Hz, NH), 7.98 (d, 2H, J 8.2 Hz, ArH), 7.87 (d, 2H, J 7.0 Hz, ArH), 7.67 (d, 2H, J 8.3 Hz, ArH), 7.56-7.44 (m, 3H, ArH), 7.16 (t, 1H, J 7.6 Hz, ArH), 6.98 (dt, 1H, J 8.1 Hz, ⁴J 1.1 Hz, ArH), 6.78 (d, 1H, J 8.0 Hz, ArH), 6.61 (t, 1H, J 7.1 Hz, ArH), 5.65 (s, 1H, C═CH), 5.32 (s, 1H, C═CH), 4.37 (d, 2H, J 5.6 Hz, NCH₂).

δ ¹³C (75 MHz): 166.5 (C═O), 165.2 (C═O), 144.1 (C═C), 143.3, 141.8, 134.6, 134.1, 131.6, 128.7, 128.3, 127.6, 127.1, 126.9, 126.0, 123.7, 116.8, 114.2 (C═C), 42.5 (C—N).

m/z (ES+, %): 371.9 (M+H, 100).

HPLC: 94% purity (eluted with MeCN/H₂O: 75/25 at 0.3 ml/min).

### Pd(0)-Catalysed 3-Component Cascade Reaction Using Piperazine Nucleophiles

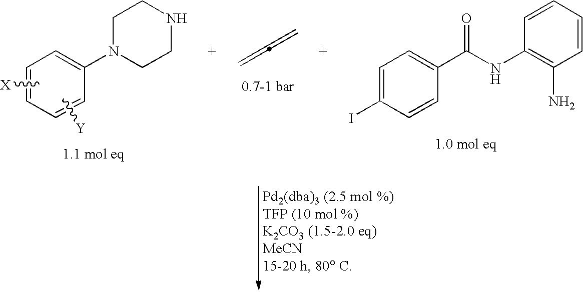

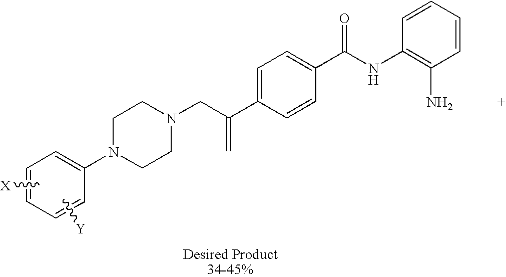

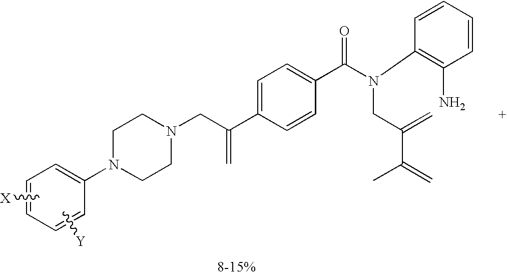

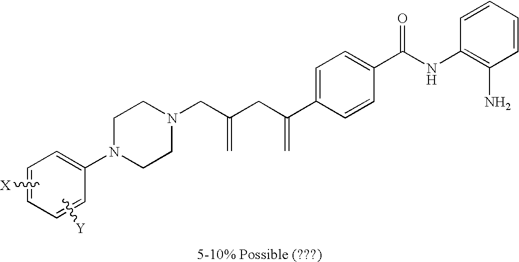

| | | Yield |
|---|---|---|
| Entry | Product | (%) |
| 1 | 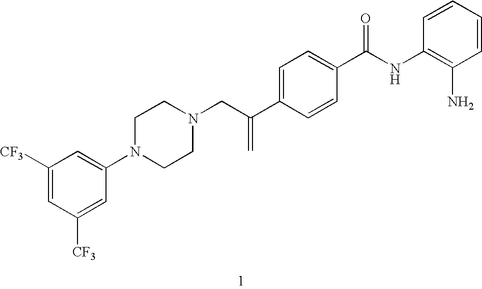 | 34 |
| 2 | 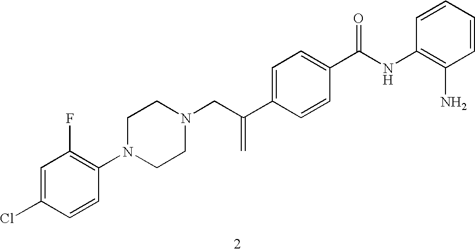 | 36 |
| 3 | 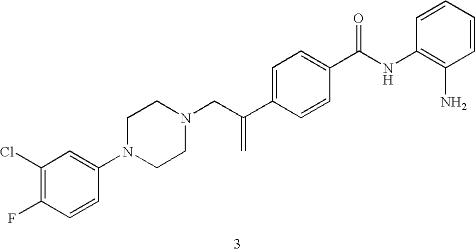 | 42 |
| 4 | 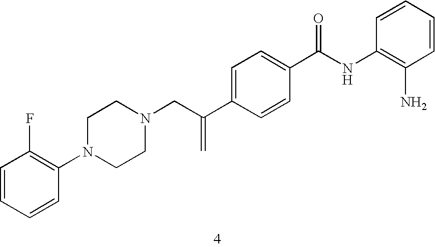 | 45 |
| 5 | 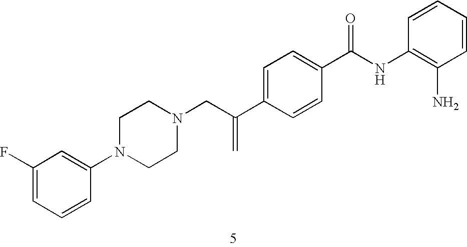 | 36 |

### N-(2-Aminophenyl)-4-[1-({4-[3,5-bis(trifluoromethyl)phenyl]piperazin-1-yl}methyl)vinyl]benzamide (1)

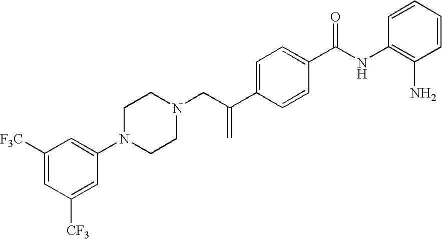

Prepared by general procedure using N-(2-amino-phenyl)-4-iodo-benzamide, (204.6 mg, 0.605 mmol), 1-(3,5-bis(trifluoromethyl)phenyl)piperazine (198.5 mg, 1.1 mol eq), potassium carbonate (167.2 mg, 2.0 mol eq), tri-2-furylphosphine (14.0 mg, 10 mol %), tris(dibenzylideneacetone) dipalladium (0) (13.8 mg, 2.5 mol %) and allene gas (1 atm, 25° C.) in acetonitrile (10 ml). The Schlenk tube was heated at 80° C. for 20 h to give the product which was purified by gradient flash chromatography, eluting with 1:1 ether-hexane and thereafter 100% ether (R_{F} 0.3) to give colourless plates (112 mg, 34%, m.p 164-166° C.).

Found: C, 60.55; H, 4.70; N, 9.85. C₂₈H₂₇N₄F₆O. 0.5H₂O requires: C, 60.35; H, 4.90; N, 10.05%.

δ ¹H (500 MHz): 7.87 (2H, d, J 8.2 Hz, ArH), 7.84 (1H, s, CONH), 7.66 (2H, d, J 8.2 Hz, ArH), 7.35 (1H, d, J 7.9 Hz, ArH), 7.26 (1H, s, ArH), 7.22 (2H, s, ArH), 7.10 (1H, t, J 7.6, ArH), 6.86-6.84 (2H, m, ArH), 5.63 (1H, s, C═CH), 5.39 (s, 1H, C═CH), 3.87 (2H, br s, NH₂), 3.45 (2H, s, NCH₂C═C), 3.26 (4H, t, J 4.9 Hz, 2×ArNCH₂CH₂), 2.65 (4H, t, J 4.9 Hz, 2×ArNCH₂CH₁).

δ ¹³C (75 MHz): 165.4 (C═O), 151.7, 143.6, 143.0, 140.6, 133.1, 132.2 (q, J_{C-F} 32.5 Hz, 2×ArC), 127.2 (2×ArC), 126.7 (2×ArC), 125.1, 124.6, 123.6 (q, J_{C-F} 272.5 Hz, 2×CF₃), 119.9, 118.5, 117.6, 114.6, 111.9 (q, J_{C-F} 3.6, 3×ArC), 62.8 (CH₂N), 52.4 (2×piperazine-CH₂N), 48.3 (2×piperazine-CH₂N).

m/z (ES+, %): 549.2 (M+H, 100).

HRMS: Found [M+H] 549.2070. C₂₈H₂₇N₄F₆O requires 549.2089.

IR (vₘₐₓ/cm⁻¹): 3582, 2978-2839, 1638 (C═O), 1621 (C═C).

### N-(2-Aminophenyl)-4-(1-{[4-(4-chloro-2-fluorophenyl)piperazin-1-yl]methyl}vinyl)benzamide (2)

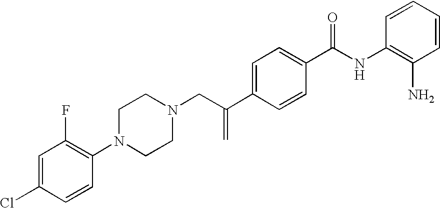

Prepared by general procedure using N-(2-amino-phenyl)-4-iodo-benzamide, (209.7 mg, 0.62 mmol), 1-(4-chloro-2-fluorophenyl)piperazine (146.4 mg, 1.1 mol eq), potassium carbonate (171.4 mg, 2.0 mol eq), tri-2-furylphosphine (14.4 mg, 10 mol %), tris(dibenzylideneacetone) dipalladium (0) (14.2 mg, 2.5 mol %) and allene gas (1 atm, 25° C.) in acetonitrile (10 ml). The Schlenk tube was heated at 80° C. for 15 h to give the product which was purified by gradient flash chromatography, eluting with 1:1 ether-hexane and thereafter 100% ether (R_{F} 0.16) to give colourless plates (102.6 mg, 36%, m.p 146-147° C.).

Found: C, 67.15; H, 5.65; N, 12.05; Cl, 7.60. C₂₆H₂₆N₄ClFO requires: C, 67.15; H, 5.65; N, 12.05; Cl, 7.60%.

δ ¹H (300 MHz): 7.96 (1H, s, CONH), 7.85 (2H, d, J 8.3 Hz, ArH), 7.64 (2H, d, J 8.3 Hz, ArH), 7.30 (1H, d, J 7.9 Hz, ArH), 7.11-6.98 (3H, m, ArH), 6.85-6.78 (3H, m, ArH), 5.60 (1H, s, C═CH), 5.37 (s, 1H, C═CH), 3.87 (2H, br s, NH₂), 3.43 (2H, s, NCH₂C═C), 3.02 (4H, t, J 4.4 Hz, 2×ArNCH₂CH₂), 2.64 (4H, t, J 4.4 Hz, 2×ArNCH₂CH₂).

δ ¹³C (75 MHz): 166.0 (C═O), 155.7 (d, J_{C-F} 249.6 Hz, ArC), 144.1, 143.6, 141.2, 139.4 (d, J_{C-F} 8.9 Hz, ArC), 133.4, 127.7 (2×ArC), 127.1 (2×ArC), 127.0, 125.8 (d, J_{C-F} 23.7, ArC), 125.0 (d, J_{C-F} 9.5, ArC), 124.9 (d, J_{C-F} 3.5 Hz, ArC), 120.2, 120.0 (2×ArC), 118.8, 117.9, 117.2 (d, J_{C-F} 24.3, ArC), 63.4 (CH₂N), 53.3 (2×piperazine-CH₂N), 50.9 (2×piperazine-CH₂N).

m/z (ES+, %): 232.7 (100), 464.8 (M⁺, 93).

IR (vₘₐₓ/cm⁻¹): 3422, 3054-2826, 1664 (C═O), 1626 (C═C).

### N-(2-Aminophenyl)-4-(1-{[4-(3-chloro-4-fluorophenyl)piperazin-1-yl]methyl}vinyl)benzamide (3)

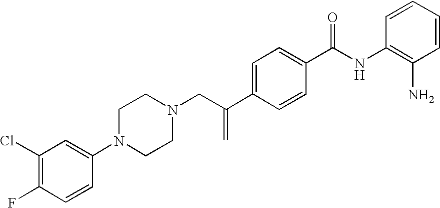

Prepared by general procedure using N-(2-amino-phenyl)-4-iodo-benzamide, (245.1 mg, 0.72 mmol), 1-(3-chloro-4-fluorophenyl)piperazine dihydrochloride (207.0 mg, 1.1 mol eq), potassium carbonate (360.6 mg, 3.6 mol eq), tri-2-furylphosphine (16.8 mg, 10 mol %), tris(dibenzylideneacetone) dipalladium (0) (16.6 mg, 2.5 mol %) and allene gas (1 atm, 25° C.) in acetonitrile (12 ml). The Schlenk tube was heated at 80° C. for 48 h to give the product which was purified by gradient flash chromatography, eluting with 1:1 ether-hexane and thereafter 100% ether (R_{F} 0.16) to give colourless plates (141.3 mg, 42%, m.p 93-95° C.).

Found: C, 67.30; H, 5.85; N, 11.95; Cl, 7.55. C₂₆H₂₆N₄ClFO requires: C, 67.15; H, 5.65; N, 12.05; Cl, 7.60%.

δ ¹H (300 MHz): 7.94 (1H, s, CONH), 7.85 (2H, d, J 8.3 Hz, ArH), 7.64 (2H, d, J 8.3 Hz, ArH), 7.30 (1H, d, J 7.8 Hz, ArH), 7.09 (1H, t, J 7.7 Hz, ArH), 7.00 (1H, t, J 8.9 Hz, ArH), 6.90-6.81 (3H, m, ArH), 6.73 (1H, m, ArH), 5.60 (1H, s, C═CH), 5.37 (s, 1H, C═CH), 3.87 (2H, br s, NH₂), 3.42 (2H, s, NCH₂C═C), 3.08 (4H, t, J 4.8 Hz, 2×ArNCHCH₂), 2.62 (4H, t, J 4.8 Hz, 2×ArNCH₂CH₂).

δ ¹³C (75 MHz): 166.0 (C═O), 152.5 (d, J_{C-F} 241.3 Hz, ArC), 148.9 (d, J_{C-F} 2.2 Hz, ArC), 144.1, 143.5, 141.1, 133.4, 127.7 (2×ArC), 127.1 (2×ArC), 126.0, 125.6, 125.0, 121.3 (d, J_{C-F} 18.0 Hz, ArC), 120.2, 118.8, 118.3, 117.9, 117.0 (d, J_{C-F} 21.7 Hz, ArC), 116.2 (d, J_{C-F} 6.1 Hz, ArC), 63.3 (CH₂N), 53.2 (2×piperazine-CH₂N), 50.0 (2×piperazine-CH₂N).

m/z (ES+, %): 465.2 (M+H, 100).

IR (vₘₐₓ/cm⁻¹): 3418, 3054-2827, 1667 (C═O), 1624 (C═C).

### N-(2-Aminophenyl)-4-(1-{[4-(2-fluorophenyl)piperazin-1-yl]methyl}vinyl)benzamide (4)

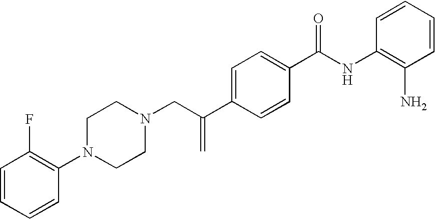

Prepared by general procedure using N-(2-amino-phenyl)-4-iodo-benzamide, (311 mg, 0.92 mmol), 1-(2-fluorophenyl)piperazine (160 μl, 1.1 mol eq), potassium carbonate (191 mg, 1.5 mol eq), tri-2-furylphosphine (21 mg, 10 mol %), tris(dibenzylideneacetone) dipalladium (0) (21 mg, 2.5 mol %) and allene gas (0.7 atm, 25° C.) in acetonitrile (15 ml). The Schlenk tube was heated at 80° C. for 18 h to give the product which was purified by gradient flash chromatography, eluting with 3:1 ether-hexane (R_{F} 0.11) to give colourless plates (180.3 mg, 45%, m.p 135-137° C.).

Found: C, 72.55; H, 6.30; N, 13.00. C₂₆H₂₇N₄FO requires: C, 72.35; H, 6.40; N, 12.80%.

δ ¹H (300 MHz): 8.08 (1H, s, CONH), 7.82 (2H, d, J 8.2 Hz, ArH), 7.61 (2H, d, J 8.2 Hz, ArH), 7.26 (1H, d, J 7.5 Hz, ArH), 7.08-7.01 (3H, m, ArH), 6.99-6.88 (2H, m, ArH), 6.81-6.77 (2H, m, ArH), 5.58 (1H, s, C═CH), 5.36 (s, 1H, C═CH), 3.87 (2H, br s, NH₂), 3.42 (2H, s, NCH₂C═C), 3.05 (4H, t, J 3.8 Hz, 2×ArNCH₂CH₂), 2.66 (4H, t, J 3.8 Hz, 2×ArNCH₂CH₂).

δ ¹³C (75 MHz): 166.1 (C═O), 156.1 (d, J_{C-F} 245.9 Hz, ArC), 144.1, 143.6, 141.3, 140.6 (d, J_{C-F} 8.4 Hz, ArC), 133.3, 127.7 (3×ArC), 127.1 (2×ArC), 125.8, 124.9, 124.8, 122.8 (d, J_{C-F} 7.9 Hz, ArC), 120.1, 119.4 (d, J_{C-F} 2.4 Hz, ArC), 118.7, 117.8, 116.5 (d, J_{C-F} 20.7 Hz, ArC), 63.4 (CH₂N), 53.5 (2×piperazine-CH₂N), 51.0 (2×piperazine-CH₂N).

m/z (ES+, %): 215.7 (100), 430.8 (M⁺, 83), 431.8 (M+H, 25).

IR (vₘₐₓ/cm⁻¹): 3420, 3069-2824, 1663 (C═O), 1623 (C═C).

### N-(2-Aminophenyl)-4-(1-{[4-(3-fluorophenyl)piperazin-1-yl]methyl}vinyl)benzamide (5)

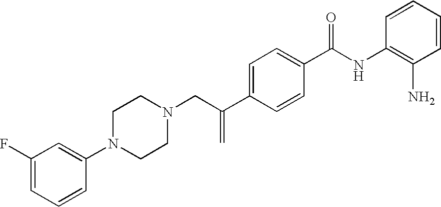

Prepared by general procedure using N-(2-amino-phenyl)-4-iodo-benzamide, (222.7 mg, 0.659 mmol), 1-(3-fluorophenyl)piperazine (130.5 mg, 1.1 mol eq), potassium carbonate (145.7 mg, 1.6 mol eq), tri-2-furylphosphine (15.3 mg, 10 mol %), tris(dibenzylideneacetone) dipalladium (0) (15.1 mg, 2.5 mol %) and allene gas (0.7 atm, 25° C.) in acetonitrile (15 ml). The Schlenk tube was heated at 80° C. for 18 h to give the product which was purified by gradient flash chromatography, eluting with 1:1 ether-hexane and thereafter 100% ether (R_{F} 0.15) to give colourless plates (100.8 mg, 36%, m.p 150-152° C.).

Found: C, 70.90; H, 6.20; N, 12.5. C₂₆H₂₇N₄FO.0.5H₂O requires: C, 71.05; H, 6.40; N, 12.75%.

δ ¹H (300 MHz): 7.91 (1H, s, CONH), 7.86 (2H, d, J 8.3 Hz, ArH), 7.65 (2H, d, J 8.3 Hz, ArH), 7.31 (1H, d, J 7.8 Hz, ArH), 7.17 (1H, q, J 7.8 Hz, ArH), 7.12-7.06 (1H, m, ArH), 6.86-6.81 (2H, m, ArH), 6.65 (1H, dd, J 8.2 and 2.0 Hz, ArH), 6.59-6.48 (2H, m, ArH), 5.61 (1H, s, C═CH), 5.37 (s, 1H, C═CH), 3.87 (2H, br s, NH₂), 3.42 (2H, s, NCH₂C═C), 3.16 (4H, t, J 4.9 Hz, 2×ArNCH₂CH₂), 2.62 (4H, t, J 4.9 Hz, 2×ArNCH₂CH₂).

δ ¹³C (75 MHz): 165.8 (C═O), 155.7 (d, J_{C-F} 249.6 Hz, ArC); 144.1, 143.5, 141.1, 133.4, 130.5 (d, J_{C-F} 10.0 Hz, ArC), 127.7 (2×ArC), 127.1 (2×ArC), 125.6, 125.0, 120.2, 118.8, 117.9, 111.5, 106.1 (d, J_{C-F} 21.3 Hz, ArC), 103.0 (d, J_{C-F} 24.9 Hz, ArC), 63.3 (CH₂N), 53.2 (2×piperazine-CH₂N), 49.0 (2×piperazine-CH₂N).

m/z (ES+, %): 236.3 (100), 430.9 (M⁺, 83), 431.8 (M+H, 25).

IR (vₘₐₓ/cm⁻¹): 3421, 2946-2825, 1664 (C═O), 1612 (C═C).

### N-(2-Aminopyridin-3-yl)-4-(1-{[(pyridin-3-ylmethyl)amino]methyl}vinyl)benzamide

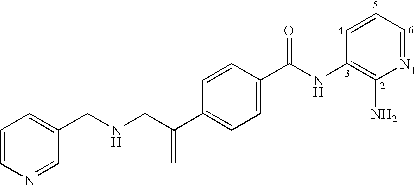

Prepared by the general procedure using N-(2-aminopyridin-3-yl)-4-iodobenzamide (0.68 g, 2.0 mmol), 3-aminomethylpyridine (0.32 g, 3.0 mmol), potassium carbonate (0.41 g, 3.0 mmol), tri-2-furylphosphine (46 mg, 10 mol %), tris(dibenzylideneacetone) dipalladium (45 mg, 2.5 mol %) and allene gas (0.5 atm). Purification by flash column chromatography eluting with 19:1 v/v EtOAc-MeOH(R_{f} 0.1) afforded the product (0.35 g, 49%) as light yellow plates, m.p 88-90° C.; vₘₐₓ/cm⁻¹ (film) 3433 and 1641; δ_{H} (500 MHz, CDCl₃) 8.52 (2H, m, ArH), 8.04 (1H, m, ArH), 7.86 (2H, d, J 8.1 Hz, ArH), 7.80 (1H, br s, CONH), 7.68 (2H, m, ArH), 7.55 (2H, d, J 8.1 Hz, ArH), 7.28 (1H, m, ArH), 6.80 (1H, dd, J 7.7 and 5.1 Hz, ArH), 5.54 and 5.39 (2×1H, 2×s, ═CH₂), 4.85 (1H, br s, NH), 3.83 (2H, s, NCH₂Ar), 3.71 (2H, s, NCH₂); δ_{C} (75 MHz, CDCl₃) 166.3 (CO), 153.7, 150.0, 148.8, 146.0, 145.7, 145.3, 143.9, 133.6, 133.2, 128.0, 127.5, 127.0, 123.8, 116.4 (CH₂), 115.0, 53.1 (CH₂N), 50.6 (CH₂N); m/z % (ES) 359 (M⁺, 100).

Analogous compounds are derived from N-(3-aminopyridin-4-yl)-, N-(4-aminopyridin-3-yl)- and N-(3-aminopyridin-2-yl)-4-iodobenzamide.

General Procedure for the Catalytic Cascade (Above) in which the Zinc Binding Group is Attached in a Final Reaction Step
General Procedure for the Catalytic C,C-Diallylation of 1,3-Dimethylbarbituric Acid Followed by Coupling Reaction with Phenylenediamine

A mixture of nucleophile (1.0 mmol, 1.0 mol equiv.), 4-iodobenzoic acid (0.2976 g, 1.2 equiv., 1.2 mmol), cesium carbonate (0.6517 g, 2.0 mol equiv.) and Pd₂(dba)₃ (0.0228 g, 2.5 mol %), TFP (0.0232 g, 10 mol %) in DMF (15 ml) was stirred for 15 min in a Schlenk tube. The reaction mixture was subjected to two freeze, pump, thaw cycles and then charged with allene (1 bar). After warming to room temperature the mixture was stirred and heated at 90-110° C. in an oil bath for 20 h, cooled to room temperature and excess allene vented. 4-(4,6-Dimethoxy-1,3,5-triazin-1-yl)-4-methyl-morpholinium chloride (1.12 g, 1.0 mol equiv) and phenylenediamine (0.1054 g, 1.5 mol equiv) were added to the reaction mixture and stirred at room temperature for 15 h. The reaction mixture then poured into water (50 ml), extracted with ethyl acetate (3×20 ml). The combined organic layer washed with aqueous sodium carbonate (15 ml), saturated ammonium chloride (15 ml) and brine (15 ml) and dried (Mg₂SO₄), filtered and the filtrate concentrated under reduced pressure. The residue was purified by flash chromatography on silica gel.

### 4,4′-[(1,3-Dimethyl-2,4,6-trioxohexahydropyrimidine-5,5-diyl)diprop-1-ene-3,2-diyl]bis[N-(2-aminophenyl)benzamide]

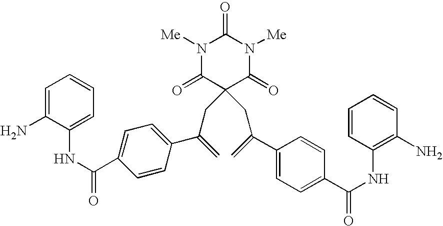

Prepared from 1,3-dimethylybarbituric acid (0.1562 g, 1.0 mmol), 4-iodobenzoic acid (0.5952 g, 2.4 mol equiv), allene (1 bar) and phenylenediamine (0.3244 g, 3.0 mol equiv) following the general procedure. Purification by flash Column chromatography eluting with ethyl acetate (R_{f} 0.10) gave the product (0.5199, 79%) as colourless solid, m.p. 161-163° C.; (Found: C, 69.15; H, 5.90; N, 12.95. C₃₈H₃₆N₆O₅ requires: C, 65.45; H, 5.60; N, 12.80%). δ_{H} (500 MHz, DMSO-d₆) 2.62 (6H, s, 2×N—CH₃), 4.88 (4H, s, 2×CH₂), 4.98 and 5.28 (2×2H, 2×s, 2× ═CH₂), 6.61 (2H, dd, J 7.5 and 7.4 Hz, ArH), 6.80 (2H, d, J 7.8 Hz, ArH), 6.99 (2H, dd, J 7.2 and 7.1 Hz, ArH), 7.17 (2H, d, J 7.6 Hz, ArH), 7.35 (4H, d, J 8.2 Hz, ArH), 7.92 (4H, d, J 8.0 Hz, ArH) and 9.67 (2H, s, 2×N—H); δ_{C} (75 MHz, DMSO-d₆) 27.9 (Me), 44.4 (CH₂), 56.2, 116.5, 116.6, 118.7, 123.5, 126.6, 126.9, 127.1, 127.9, 134.1, 142.9, 143.2, 143.5, 150.1, 164.9 (CO) and 169.9 (CO); m/z (ES) (%) 657.3 (60, M⁺+H), 328.9 (100) etc; νₘₐₓ/cm⁻¹ (solid) 3469, 3374, 3221, 3056, 1744, 1675, 1504, 1380, 1307, 1264, 1188, 1158, 1133, 1087, 1046, 1014, 907, 868 and 751.

### N-(2-Aminophenyl)-4-{1-[(3-methyl-2,5-dioxoimidazolidin-]-yl)methyl]vinyl}benzamide

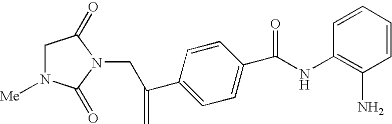

Prepared from 1-methylhydantoin (0.1176 g, 1.0 mmol), 4-iodobenzoic acid (0.2976 g, 1.2 equiv), allene (1 bar) and phenylenediamine (0.1622 g, 1.5 mol equiv) following the general procedure. Purification by flash Column chromatography eluting with ethyl acetate (R_{f} 0.17) gave the product (0.2156 g, 59%) as colourless solid, m.p. 179-181° C.; (Found: C, 65.90; H, 5.70; N, 15.20. C₂₀H₂₀N₄O₃ requires: C, 65.90; H, 5.55; N, 15.35%). δ_{H} (500 MHz, DMSO-d₆) 2.88 (3H, s, N-Me), 4.04 (2H, s, N—CH₂), 4.44 (2H, s, CO—CH₂), 4.91 (2H, s, NH₂), 5.17 and 5.62 (2×1H, 2×s, ═CH₂), 6.62 (1H, t, J 7.3 Hz, ArH), 6.81 (1H, d, J 7.8 Hz, ArH), 6.99 (1H, t, J 7.4 Hz, ArH), 7.19 (1H, d, J 7.5 Hz, ArH), 7.66 (2H, d, J 8.0 Hz, ArH) and 7.99 (2H, d, J 7.8 Hz, ArH), 9.69 (1H, s, CO—NH); δ_{C} (75 MHz, DMSO-d₆) 29.6 (Me), 41.4 (CH₂), 51.6 (CH₂), 114.6 (═CH₂), 116.4, 116.6, 123.5, 126.0, 126.9, 127:1, 128.3, 134.3, 141.0, 141.2, 143.6, 156.5 (CO), 165.2 (CO) and 170.6 (CO); m/z (ES) (%) 365.0 (100, M⁺+H); νₘₐₓ/cm⁻¹ (solid) 3417, 3317, 3058, 2957, 2924, 1774, 1696, 1641, 1591, 1530, 1488, 1454, 1381, 1332, 1303, 1265, 1243, 1147, 1016, 973, 936, 913, 888, 857, 780, 748 and 721.

### N-(2-aminophenyl)-4-{1-[(2-chloro-10H-phenothiazin-10-yl)methyl]vinyl}benzamide

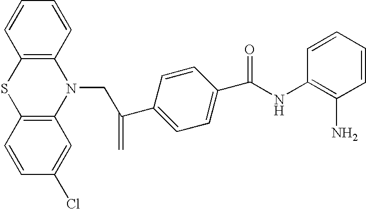

Prepared from 2-chlorophenothiazine (0.2337 g, 1.0 mmol), 4-iodobenzoic acid (0.2976 g, 1.2 equiv), allene (1 bar) and phenylenediamine (0.1622 g, 1.5 mol equiv) following the general procedure. Purification by flash Column chromatography eluting with 3:7 (v/v) petroleum ether-diethyl ether (R_{f} 0.18) gave the product (0.3396 g, 69%) as colourless solid, m.p. 126-128° C.; δ_{H} (500 MHz, DMSO-d₆) 4.97 (2H, s, NH₂), 5.00 (2H, s, CH₂), 5.23 and 5.76 (2×1H, 2×s, ═CH₂), 6.62 (1H, t, J 7.3 Hz, ArH), 6.81 (1H, d, J 7.5 Hz, ArH), 6.92-7.01 (5H, m, ArH), 7.08-7.13 (2H, m, ArH), 7.16-21 (2H, m, ArH), 7.73 (2H, d, J 7.9 Hz, ArH), 8.02 (2H, d, J 8.1 Hz, ArH), 9.71 (1H, s, CO—NH); δ_{C} (75 MHz, DMSO-d₆) 51.7 (CH₂), 115.7 (═CH₂), 116.2, 116.5, 116.6, 121.6, 122.2, 122.6, 123.5, 123.6, 126.3, 126.9, 127.1, 128.0, 128.1, 128.3, 132.5, 134.3, 140.2, 141.2, 143.5, 143.6, 145.5 and 165.1 (CO); m/z (ES) (%) 384.0 (100, M⁺); vₘₐₓ/cm⁻¹ (solid) 3400, 3289, 3071, 2926, 1627, 1566, 1538, 1493, 1462, 1410, 1341, 1308, 1276, 1249, 1223, 1159, 1129, 1103, 1015, 912, 855 and 777.

### N-(2-Aminophenyl)-4-{1-[(9-oxoacridin-10(9H)-yl)methyl]vinyl}benzamide (rp-207 [coupling])

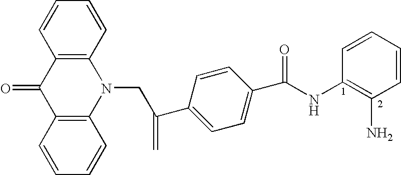

Prepared from 9(10H)-acridone (0.1952 g, 1.0 mmol), 4-iodobenzoic acid (0.2976 g, 1.2 equiv), allene (1 bar) and 1,2-phenylenediamine (0.1054 g, 1.5 mol equiv) following the general procedure. Purification by flash Column chromatography eluting with 2:3 v/v petroleum ether-ethyl acetate (R_{f} 0.22) gave the product (0.2457 g, 54%) as pale yellow solid, m.p. 167-160° C.; δ_{H} (500 MHz, DMSO-d₆) 4.66 and 5.65 (2×1H, 2×s, ═CH₂), 4.96 (2H, s, NH₂), 5.56 (2H, s, CH₂), 6.65 (1H, t, J 7.3 Hz, ArH), 6.85 (1H, d, J 7.8 Hz, ArH), 7.02 (1H, t, J 7.4 Hz, ArH), 7.25 (1H, d, J 7.3 Hz, ArH), 7.37 (2H, t, J 7.3 Hz, ArH), 7.59 (2H, d, J 8.7 Hz, ArH), 7.81 (2H, t, J 7.5 Hz, ArH), 7.91 (2H, d, J 7.8 Hz, ArH), 8.13 (2H, d, J 7.5 Hz, ArH), 8.40 (2H, d, J 7.8 Hz, ArH) and 9.79 (1H, s, CO—NH); δ_{C} (75 MHz, DMSO-d₆) 49.8 (CH₂), 113.3 (═CH₂), 114.8, 116.6, 121.9, 122.0, 126.6, 126.3, 126.9, 127.2, 128.5, 131.7, 134.6, 134.7, 139.8, 140.1, 140.9, 141.3, 142.2, 143.6, 165.2 (CO) and 177.1 (CO); m/z (ES) (%) 446.0 (100, M⁺+H); νₘₐₓ/cm⁻¹ (solid) 3312, 3060, 2642, 2340, 2181, 1927, 1821, 1466, 1374, 1215, 1171, 1135, 1050, 1015, 963, 932, 909, 852, 803 and 750.

### N-(2-Aminophenyl)-4-(J-[(6-oxophenanthridin-5(6H)-yl)methyl]vinyl}benzamide

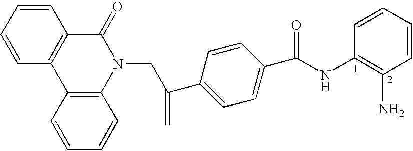

Prepared from 6(5H)-phenanthridinone (0.2034 g, 1.0 mmol), 4-iodobenzoic acid (0.2976 g, 1.2 equiv), allene (1 bar) and 1,2-phenylenediamine (0.1622 g, 1.5 mol equiv) following the general procedure. Purification by flash Column chromatography eluting with 1:4 v/v petroleum ether-ethyl acetate (R_{f} 0.17) gave the product (0.2747 g, 61%) as colourless solid, m.p. >220° C.; δ_{H} (500 MHz, DMSO-d₆) 4.75 and 5.58 (2×1H, 2×s, ═CH₂), 4.93 (2H, s, NH₂), 5.47 (2H, s, CH₂), 6.62 (1H, dd, J 7.6 and 7.3 Hz, ArH), 6.81 (1H, d, J 7.9 Hz, ArH), 6.99 (1H, dd, J 7.8 and 7.5 Hz, ArH), 7.20 (1H, d, J 7.6 Hz, ArH), 7.37-7.41 (2H, m, ArH), 7.59 (1H, dd, J 7.9 and 7. Hz, ArH), 7.70 (1H, t, J 7.5 Hz, ArH), 7.80 (2H, d, J 8.2 Hz, ArH), 7.91 (1H, dd, J 7.9 and 7.3 Hz, ArH), 8.05 (2H, d, J 8.2 Hz, ArH), 8.43 (1H, d, J 7.7 Hz, ArH), 8.55 (1H, d, J 7.7 Hz, ArH), 8.60 (1H, d, J 8.2 Hz, ArH) and 9.72 (1H, s, CO—NH); δ_{C} (75 MHz, DMSO-d₆) 45.6 (CH₂), 113.1 (═CH₂), 116.5, 116.6, 118.9, 122.9, 123.1, 123.6, 124.1, 124.9, 126.1, 126.9, 127.1, 128.4, 128.6, 128.7, 130.3, 133.5, 133.8, 134.5, 137.1, 141.1, 141.4, 143.5, 160.7 (CO) and 165.2 (CO); m/z (ES) (%) 446.0 (100, M⁺+H); νₘₐₓ/cm⁻¹ (solid) 3345, 3097, 2731, 2367, 2127, 1953, 1825, 1477, 1382, 1212, 1171, 1121, 1078, 1032, 969, 934, 914, 858, 806 and 732.

### N-(2-aminophenyl)-4-(1-{[(11aR)-5,11-dioxo-2,3,11,11a-tetrahydro-1H-pyrrolo[2,1-c][1,4]benzodiazepin-10(5H)-yl]methyl}vinyl)benzamide

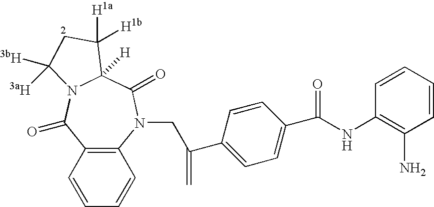

Prepared from (S)-(+)-2,3-dihydro-1H-pyrrolo[2,1-c]benzodiazepine-5,11(10H,11aH)-dione (0.2162 g, 1.0 mmol), 4-iodobenzoic acid (0.2976 g, 1.2 equiv), allene (1 bar) and phenylenediamine (0.1622 g, 1.5 mol equiv) following the general procedure. Purification by flash Column chromatography eluting with 9:1 (v/v) diethyl ether-methanol (R_{f} 0.20) gave the product (0.3255 g, 70%) as colourless solid, m.p. 160-162° C.; (Found: C, 71.80; H, 5.75; N, 11.95. C₂₈H₂₆N₄O₃ requires: C, 72.10; H, 5.60; N, 12.00%). δ_{H} (500 MHz, DMSO-d₆) 1.87-2.00 (3H, m, H-2 and H-1a), 2.49 (1H, m, H-1b), 3.32 (1H, m, H-3a), 3.43 (1H, m, H-3b), ------------------6.62 (1H, t, J 7.3 Hz, ArH), 6.80 (1H, d, J 8.0 Hz, ArH), 6.99 (1H, t, J 7.2 Hz, ArH), 7.20 (1H, d, J 7.6 Hz, ArH), 7.31 (1H, m, ArH), 7.40 (2H, d, J 8.4 Hz, ArH), 7.56-7.61 (2H, m, ArH), 7.66 (1H, d, J 7.9 Hz, ArH), 7.92 (2H, d, J 8.1 Hz, ArH), 9.62 (1H, s, CO—NH); δ_{C} (75 MHz, DMSO-d₆) 22.1 (CH₂), 25.2 (CH₂), 44.9 (CH₂), 48.3 (CH₂), 55.47, 113.9 (═CH₂), 114.9, 115.1, 122.1, 122.2, 124.3, 124.4, 125.3, 125.5, 126.5, 128.3, 129.3, 130.6, 132.6, 137.2, 139.7, 140.9, 141.9, 162.5, 163.6 (CO) and 168.1 (CO); m/z (ES) (%) 466.0 (100, M⁺); vₘₐₓ/cm⁻¹ (solid) 3425, 3063, 2957, 1901, 1845, 1679, 1531, 1301, 1245, 1206, 1165, 1121, 1090, 1051, 982, 920, 864 and 764.

Screening of Benzamide Derivatives Against Cancer Cell Lines Tables 1 and 2 tabulate by way of example the results obtained for compounds (4) and (6). GI50 shows the concentration which inhibits cancer cell growth by 50%. TGI (total growth inhibition) shows the concentration which completely inhibits growth. LC50 shows the concentration which kills 50% of the original cancer cells.

TABLE 1
Summary of activity of compound (4). ‘—’ indicates
a concentration exceeding 50 μM.

| | | GI50 | TGI | LC50 |
|---|---|---|---|---|
| Cancer Type | Cell Line | (nM) | (μM) | (μM) |
| Leukaemia | RPMI-8226 | 510 | — | — |
| | SR | 484 | — | — |
| Non-Small | NCI-H23 | 178 | 1.5 | 11.4 |
| Cell Lung | NCI-H522 | 220 | 1.4 | 20.8 |
| Colon | HCC-2998 | 90 | 0.4 | 2.0 |
| | HCT-15 | 836 | 7.8 | — |
| Melanoma | LOX IMVI | 99 | 0.6 | 6.4 |
| | SK-MEL-28 | 389 | 6.5 | — |
| | UACC-257 | 499 | 15.5 | — |
| | UACC-62 | 666 | 2.9 | 20.7 |
| Ovarian | IGROV1 | 245 | 10.5 | — |
| | OVCAR-8 | 768 | 12.5 | — |
| Renal | ACHN | 844 | 2.8 | 14.4 |
| | RXF 393 | 102 | 0.6 | 46.0 |
| | SN12C | 629 | 2.7 | 27.0 |
| Breast | NCI/ADR-RES | 613 | 2.0 | — |
| | MDA-MB-231/ATCC | 748 | 2.7 | 19.2 |
| | MDA-MB-435 | 98 | 0.6 | ~4.0 |

TABLE 2
Summary of activity of compound (6). ‘—’ indicates
a concentration exceeding 50 μM.

| | | GI50 | TGI | LC50 |
|---|---|---|---|---|
| Cancer Type | Cell Line | (nM) | (μM) | (μM) |
| Leukaemia | SR | 622 | — | — |
| Non-Small | NCI-H23 | 1040 | 3.2 | 13.4 |
| Cell Lung | NCI-H522 | 150 | 3.0 | — |
| Colon | HCC-2998 | 731 | 1.9 | 5.0 |
| Melanoma | LOX IMVI | 763 | 2.4 | — |
| Ovarian | IGROV1 | 32 | 8.5 | — |
| Breast | NCI/ADR-RES | 973 | 3.9 | 18.3 |
| | MDA-MB-435 | 948 | 2.2 | 5.6 |

All of the cell lines against which compound (6) has nanomolar activity are also potently inhibited by compound (4). Compound (4) shows additional potency in some melanoma, ovarian, renal and breast cell lines.

However, we have especially found the compounds of the invention are efficacious in the treatment of cancers selected from colonic cancer, melanoma and non-small cell lung cancer.

### Comparison Tests of Compound (4) Versus MS275

### Colon Cancer

6 colon cancer cell lines were screened against both compound (4) and MS275. Compound (4) showed >2-fold potency over MS275 in 3 cell lines.

| | Compound (4) (μm) | MS275 (μm) |
|---|---|---|
| HCC-2998 | 0.09 | 1.26 |
| HCT-15 | 0.84 | 3.98 |
| KM-12 | 1.15 | 25.0 |

### Non-Small Cell Lung Cancer

8 non-small cell lung cancer cell lines were screened against both compound (4) and MS275. Compound (4) showed >2-fold potency over MS275 in 2 cell lines.

| | Compound (4) (μm) | MS275 (μm) |
|---|---|---|
| NCI-H23 | 0.18 | 1.03 |
| NCI-H522 | 0.22 | 1.00 |

### Renal Cancer

7 renal cancer cell lines were screened against both compound (4) and MS275. Compound (4) showed >2-fold potency over MS275 in 4 cell lines.

| | Compound (4) (μm) | MS275 (μm) |
|---|---|---|
| RXF 393 | 0.10 | 2.00 |
| SN12C | 0.63 | 5.01 |
| ACHN | 0.84 | 2.00 |
| 786-0 | 1.05 | 3.16 |

### Melanoma Cancer

7 melanoma cancer cell lines were screened against both compound (4) and MS275. Compound (4) showed >2-fold potency over MS275 in 3 cell lines.

| | Compound (4) (μm) | MS275 (μm) |
|---|---|---|
| LOX IMVI | 0.09 | 0.79 |
| SK-MEL-28 | 0.39 | 2.00 |
| UACC-257 | 0.50 | 1.54 |

### Ovarian Cancer

6 ovarian cancer cell lines were screened against both compound (4) and MS275. Compound (4) showed >2-fold potency over MS275 in 3 cell lines.

| | Compound (4) (μm) | MS275 (μm) |
|---|---|---|
| IGROV1 | 0.25 | 2.51 |
| SK-OV-3 | 1.08 | 5.01 |
| OVCAR-4 | 1.91 | 20.0 |

### Breast Cancer

7 breast cancer cell lines were screened against both compound (4) and MS275. Compound (4) showed >2-fold potency over MS275 in 3 cell lines.

| | Compound (4) (μm) | MS275 (μm) |
|---|---|---|
| MDA-MB-435 | 0.09 | 10.0 |
| NCI/ADR-RES | 0.61 | 1.26 |
| MDA-MB-231/ATCC | 0.75 | 2.00 |

1. A compound of formula (II); 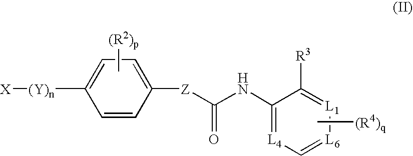
wherein:
the group X hereinafter referred to as the CAP group is a compound of general formula (III) or (IV); 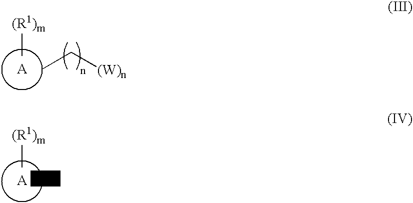
W is carbon, —CH—, —CH₂—, nitrogen, sulphur, oxygen, —N(R^{a})—, —C(O)O—, —C(O)—, —N(R^{a})C(O)—, —N(R^{a})C(O)N(R^{b})—, —N(R^{a})C(O)O—, —OC(O)N(R^{a})—, —C(O)N(R^{a})—, S(O)ᵣ—, —SO₂N(R^{a})—, —N(R^{a})SO₂—, —N(R^{a})C(S)N(R^{b})—, —N(R^{a})C(S)O—, —C(S)— or —C(S)N(R^{a})—; wherein R^{a} and R^{b} are independently selected from hydrogen or C₁₋₆alkyl optionally substituted by one or more V and r is 0-2;
n is 1, 2, 3, 4, 5 or 6;
Ring A is an optionally substituted carbocyclyl or heterocyclyl group wherein each substitutable carbon or heteroatom in Ring A is optionally and independently substituted by one or more of halo, C₁₋₆ alkyl, carbocyclyl or heterocyclyl; and wherein if Ring A contains an —NH— moiety other than W that nitrogen may be optionally substituted by a group selected from K;
L is carbon or nitrogen;
R¹ is a substituent on carbon and is selected from oxygen, halo, nitro, cyano, hydroxy, trifluoromethyl, trifluoromethoxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N—(C₁₋₆alkyl)amino, N,N—(C₁₋₆alkyl)₂amino, C₁₋₆alkanoylamino, N—(C₁₋₆alkyl)carbamoyl, N,N—(C₁₋₆alkyl)₂carbamoyl, C₃₋₈cycloalkyl, C₃₋₈cycloalkylC₁₋₆alkyl, C₁₋₆alkylS(O)ₐ wherein a is 0 to 2, C₁₋₆alkoxycarbonyl, N—(C₁₋₆alkyl)sulphamoyl, N,N—(C₁₋₆alkyl)₂sulphamoyl, aryl, aryloxy, arylC₁₋₆alkyl, heterocyclic group, (heterocyclic group)C₁₋₆alkyl, or a group (D-E-); wherein R¹, including group (D-E-), is optionally substituted on carbon by one or more T; and wherein if said heterocyclic group contains an —NH— moiety that nitrogen is optionally substituted by J;
T is halo, nitro, cyano, hydroxy, trifluoromethyl, trifluoromethoxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N—(C₁₋₆alkyl)amino, N,N—(C₁₋₆alkyl)₂amino, C₁₋₆alkanoylamino, N—(C₁₋₆alkyl)carbamoyl, N,N—(C₁₋₆alkyl)₂carbamoyl, C₁₋₆alkylS(O)ₐ wherein a is 0 to 2, C₁₋₆alkoxycarbonyl, N—(C₁₋₆alkyl)sulphamoyl, N,N—(C₁₋₆alkyl)₂sulphamoyl, or a group (D′-E′-); wherein T, including group (D′-E′-), is optionally substituted on carbon by one or more R;
R and Q are independently selected from halo, nitro, cyano, hydroxy, trifluoromethyl, trifluoromethoxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N—(C₁₋₆alkyl)amino, N,N—(C₁₋₆alkyl)₂amino, C₁₋₆alkanoylamino, N—(C₁₋₆alkyl)carbamoyl, N,N—(C₁₋₆alkyl)₂carbamoyl, C₁₋₆alkylS(O)ₐ wherein a is 0 to 2, C₁₋₆alkoxycarbonyl, N—(C₁₋₆alkyl)sulphamoyl, N,N—(C₁₋₆alkyl)₂sulphamoyl;
G, J and K are independently selected from C₁₋₈alkyl, C₂₋₈alkenyl, C₂₋₈alkynyl, C₁₋₈alkanoyl, C₁₋₈alkylsulphonyl, C₁₋₈alkoxycarbonyl, carbamoyl, N—(C₁₋₈alkyl)carbamoyl, N,N—(C₁₋₈alkyl)₂carbamoyl, benzyloxycarbonyl, benzoyl, phenylsulphonyl, aryl, arylC₁₋₆alkyl or (heterocyclic group)C₁₋₆alkyl; wherein G, J and K are optionally substituted on carbon by one or more P; and wherein if said heterocyclic group contains an —NH— moiety that nitrogen is optionally substituted by hydrogen or C₁₋₆alkyl;
P is halo, nitro, cyano, hydroxy, trifluoromethyl, trifluoromethoxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N—(C₁₋₆alkyl)amino, N,N—(C₁₋₆alkyl)₂amino, C₁₋₆alkanoylamino, N—(C₁₋₆alkyl)carbamoyl, N,N—(C₁₋₆alkyl)₂carbamoyl, C₁₋₆alkylS(O)ₐ wherein a is 0 to 2, C₁₋₆alkoxycarbonyl, C₁₋₆alkoxycarbonylamino, N—(C₁₋₆alkyl)sulphamoyl, N,N—(C₁₋₆alkyl)₂sulphamoyl, aryl, aryloxy, arylC₁₋₆alkyl, arylC₁₋₆alkoxy, heterocyclic group, (heterocyclic group)C₁₋₆alkyl, (heterocyclic group)C₁₋₆alkoxy, or a group (D″-E″-); wherein P, including group (D″-E″-), is optionally substituted on carbon by one or more Q;
D, D′ and D″ are independently selected from C₁₋₆alkyl, C₂₋₆alkenyl, C₂₋₆alkynyl, C₃₋₈cycloalkyl, C₃₋₈cycloalkylC₁₋₆alkyl, aryl, arylC₁₋₆alkyl, heterocyclic group, (heterocyclic group)C₁₋₆alkyl, phenyl or phenylC₁₋₆alkyl wherein D, D′ and D″ are optionally substituted on carbon by one or more M; and wherein if said heterocyclic group contains an —NH— moiety that nitrogen is optionally substituted by a group selected from G;
E, E′ and E″ are independently selected from —N(R^{a})—, —O—, —C(O)O—, —C(O)—, —N(R^{a})C(O)—, —N(R^{a})C(O)N(R^{b})—, —N(R^{a})C(O)O—, —OC(O)N(R^{a})—, —C(O)N(R^{a})—, S(O)ᵣ—, —SO₂N(R^{a})—, —N(R^{a})SO₂—; wherein R^{a} and R^{b} are independently selected from hydrogen or C₁₋₆alkyl optionally substituted by one or more V and r is 0-2;
M and V are independently selected from halo, nitro, cyano, hydroxy, trifluoromethyl, trifluoromethoxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N—(C₁₋₆alkyl)amino, N,N—(C₁₋₆alkyl)₂amino, C₁₋₆alkanoylamino, N—(C₁₋₆alkyl)carbamoyl, N,N—(C₁₋₆alkyl)₂carbamoyl, C₁₋₆alkylS(O)ₐ wherein a is 0 to 2, C₁₋₆alkoxycarbonyl, N—(C₁₋₆alkyl)sulphamoyl or N,N—(C₁₋₆alkyl)₂sulphamoyl;
m is 0, 1, 2, 3 or 4; wherein the values of R¹ are the same or different;
wherein Y is an unsaturated group;
R² is absent or halo;
p is 0, 1 or 2; wherein the values of R² are the same or different;
Z is absent, a direct carbon-carbon bond, C₁₋₆alkyl, C₂₋₆alkenyl, C₂₋₆alkynyl or —C(R^{c})═N—O—; wherein R^{c} is independently selected from hydrogen or C₁₋₆alkyl optionally substituted by one or more V and r is 0-2;
R³ is absent, amino or hydroxy;
R⁴ is halo, nitro, cyano, hydroxy, trifluoromethyl, trifluoromethoxy, amino, carboxy, carbamoyl, mercapto, sulphamoyl, C₁₋₃alkyl, C₂₋₃alkenyl, C₂₋₃alkynyl, C₁₋₃alkoxy, C₁₋₃alkanoyl, C₁₋₃alkanoyloxy, N—(C₁₋₃alkyl)amino, N,N—(C₁₋₃alkyl)₂amino, C₁₋₃alkanoylamino, N—(C₁₋₃alkyl)carbamoyl, N,N—(C₁₋₃alkyl)₂carbamoyl, C₁₋₃alkylS(O)ₐ wherein a is 0 to 2, C₁₋₃alkoxycarbonyl, N—(C₁₋₃alkyl)sulphamoyl or N,N—(C₁₋₃alkyl)₂sulphamoyl;
q is 0, 1 or 2; wherein the values of R⁴ may be the same or different;
or a pharmaceutically acceptable salt or in vivo hydrolysable ester or amide thereof.

2. A compound of the formula (II) as claimed in claim 1 wherein Y is the 2-propylene derivative (V), the optionally functionalised derivative of the double bond in (V) or the reduced 2-propyl product (VI) 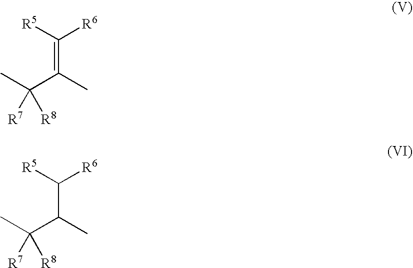
wherein R⁵, R⁶, R⁷ and R⁸ are independently selected from hydrogen, halo, C₁₋₆alkyl, C₃₋₈cycloalkyl, C₃₋₈cycloalkylC₁₋₆alkyl, aryl, arylC₁₋₆alkyl, heterocyclic group, (heterocyclic group)C₁₋₆alkyl, phenyl, phenylC₁₋₆alkyl, C₁₋₆alkoxy, C₁₋₆alkanoyl, C₁₋₆alkanoyloxy, N—(C₁₋₆alkyl)amino, N,N—(C₁₋₆alkyl)₂amino, C₁₋₆alkanoylamino, N—(C₁₋₆alkyl)carbamoyl, N,N—(C₁₋₆alkyl)₂carbamoyl, C₁₋₆alkylS(O)ₐ wherein a is 0 to 2, C₁₋₆alkoxycarbonyl, N—(C₁₋₆alkyl)sulphamoyl or N,N—(C₁₋₆alkyl)₂sulphamoyl;

3. A compound of the formula (II) as claimed in claim 1 wherein Ring A aryl, arylC₁₋₆alkyl, heterocyclic group, (heterocyclic group)C₁₋₆alkyl, phenyl, phenylC₁₋₆alkyl, pyridyl, quinolyl, indolyl, pyrimidinyl, morpholinyl, piperidinyl, piperazinyl, pyridazinyl, pyrazinyl, thiazolyl, thienyl, thienopyrimidinyl, thienopyridinyl, purinyl, 1′,2′,3′,6′-tetrahydropyridinyl, triazinyl, oxazolyl, pyrazolyl, furanyl or tetrahydro-β-carbolinyl; wherein Ring A is optionally and independently substituted by one or more of halo, C₁₋₆ alkyl, carbocyclyl or heterocyclyl; and wherein if Ring A contains an —NH— moiety other than W that nitrogen is optionally substituted by a group selected from K.

4. A compound of the formula (II) as claimed in claim 1 wherein Ring A is pyridine-4-yl, pyridine-3-yl, pyridine-2-yl; morpholin-4-yl; piperidin-4-yl, piperidin-3-yl, piperidin-2-yl; piperazin-4-yl, thiazol-2-yl, thien-2-yl, furan-3-yl, pyrrolidin-1-yl, piperidin-1-yl; triazol-1-yl or 1′,2′,3′,6′-tetrahydropyridin-4-yl wherein if Ring A contains an —NH— moiety that nitrogen is optionally substituted by a group selected from K.

5. A compound of the formula (II) as claimed in claim 1 wherein the compound is selected from the group consisting of

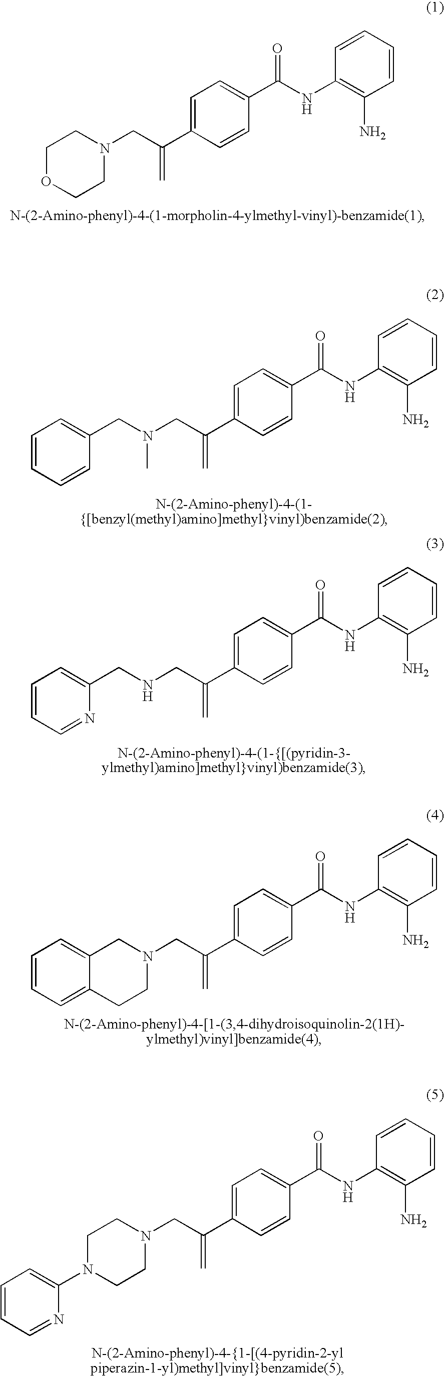

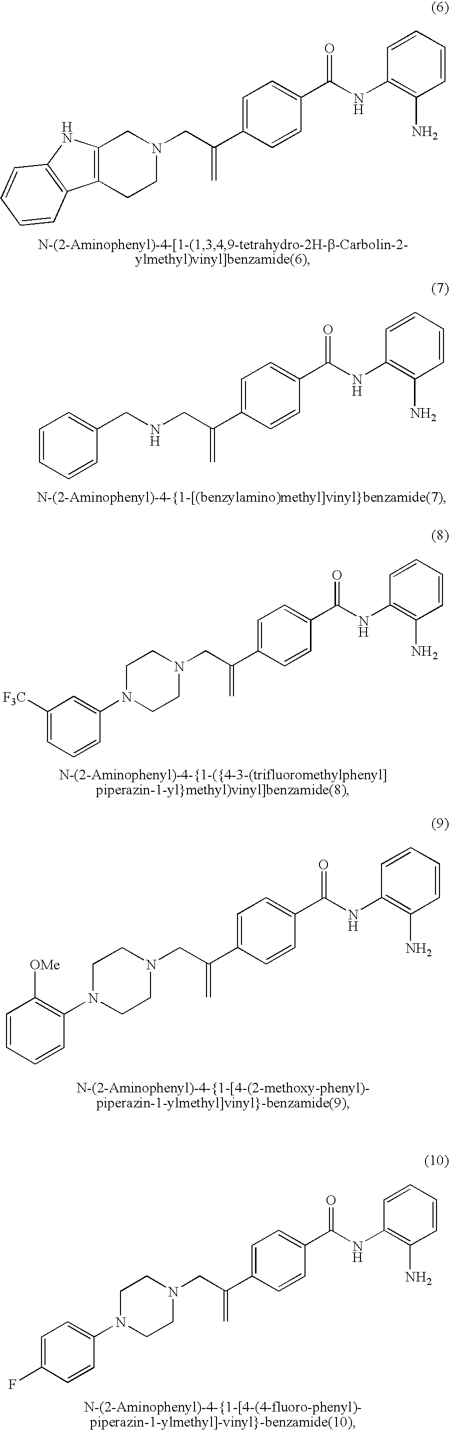

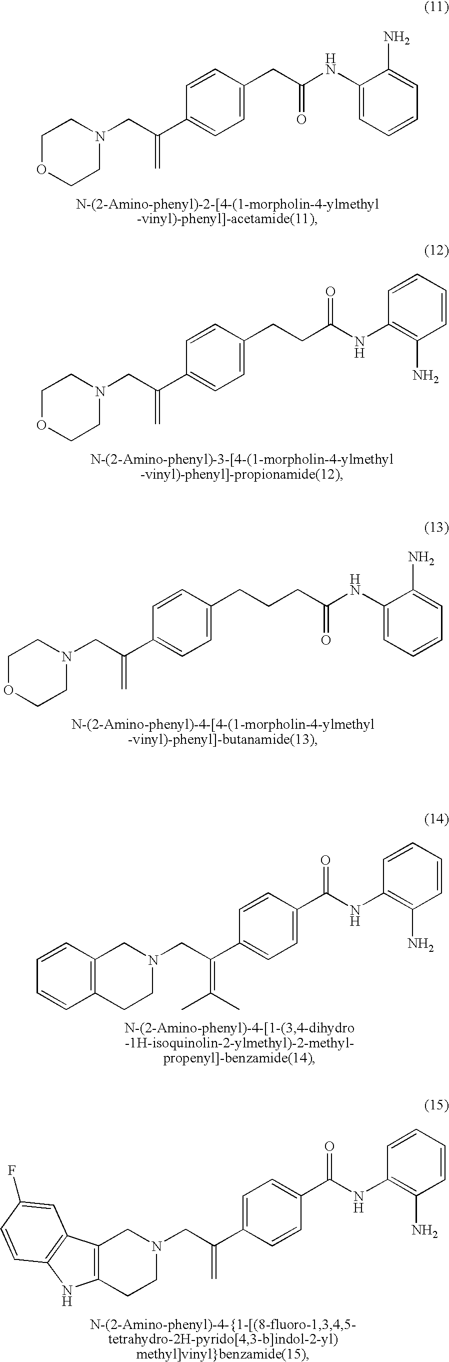

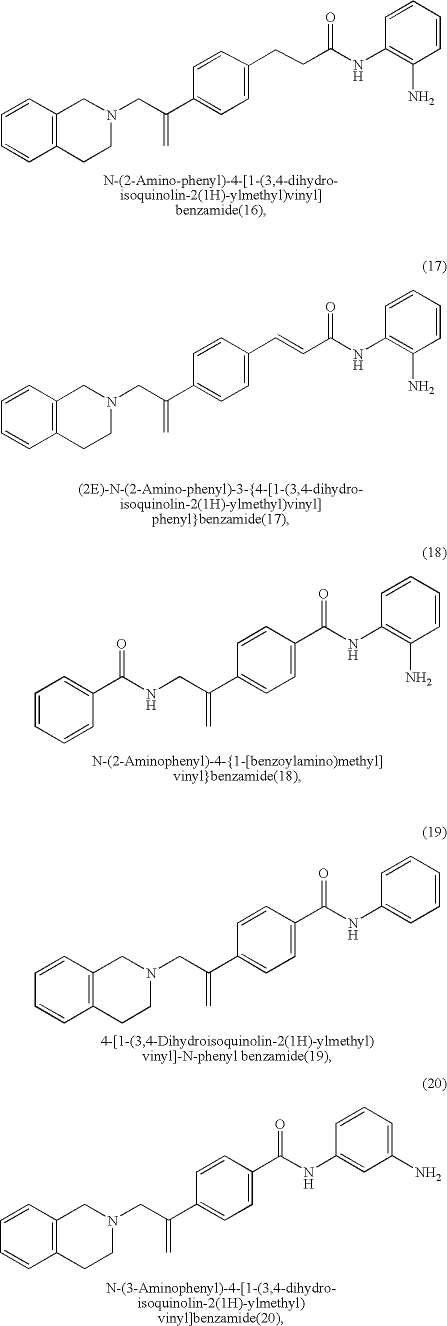

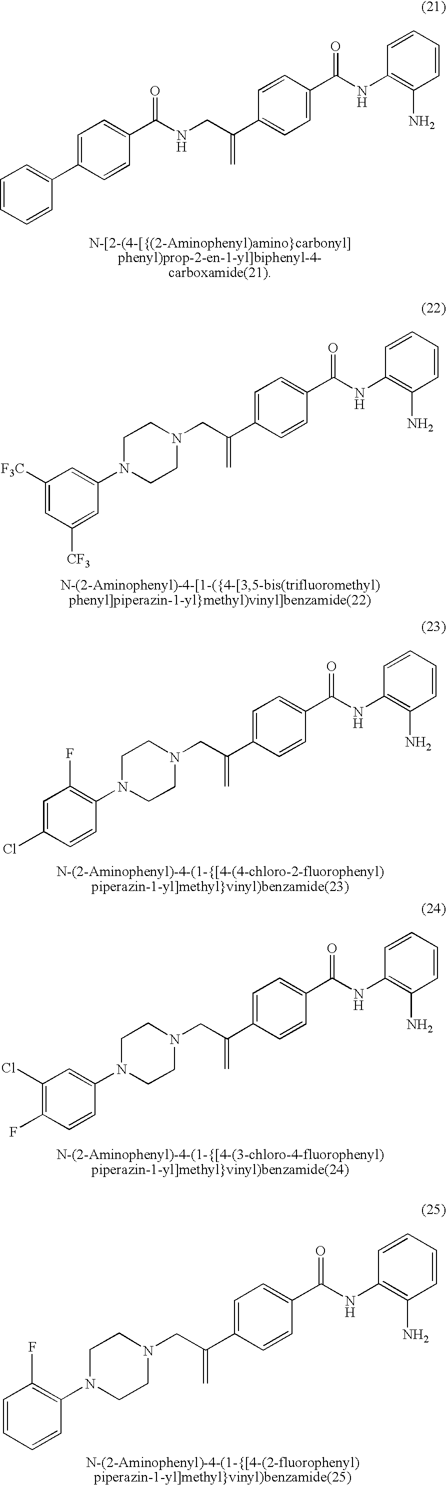

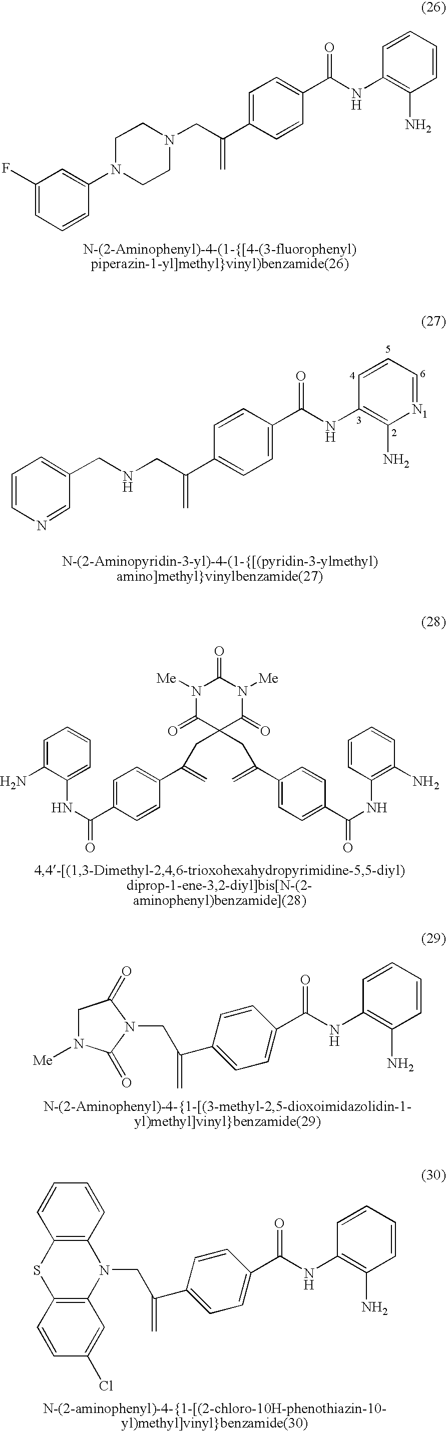

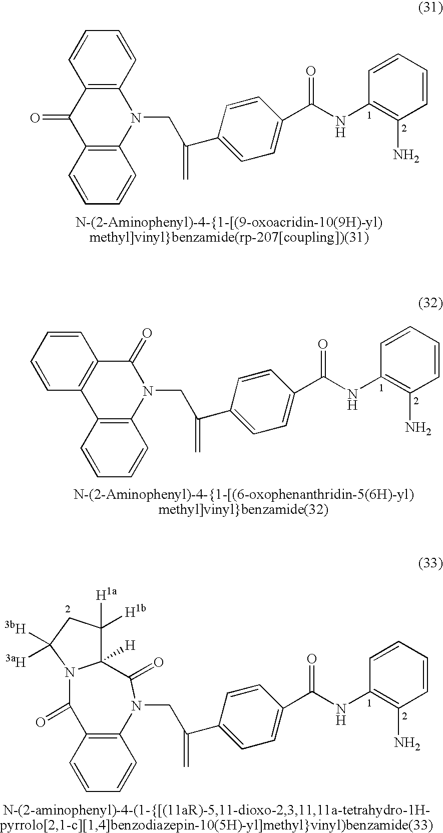

6. A compound of the formula (II) as claimed in claim 1 wherein the compound is selected from the group consisting of N-(2-amino-phenyl)-4-[1-(3,4-dihydroisoquinolin-2(1H)-ylmethyl)vinyl]benzamide (4), N-(2-aminophenyl)-4-[1-(1,3,4,9-tetrahydro-2H-β-carbolin-2-ylmethyl)vinyl]benzamide (6) and N-(2-aminophenyl)-4-{1-({4-3-(trifluoromethylphenyl)piperazin-1-yl}methyl)vinyl]benzamide (8).

7. A pharmaceutical composition comprising the compound of the formula (II), or a pharmaceutically acceptable salt or an in vivo hydrolysable ester or amide thereof, as claimed in any of claims 1 to 6 in association with a pharmaceutically acceptable carrier or diluent.

8. A process for the manufacture of a compound of formula II, or a pharmaceutically acceptable salt or an in vivo hydrolysable ester or amide thereof, according to claim 1, which process comprises reacting the following compounds in the presence of a catalyst:
i) a nucleophile of formula (III) or (IV) wherein the nucleophilic group is either W or is located within R¹;
ii) allene gas or a substituted allene; and
iii) a halogen or triflate substituted aryl molecule of formula (VII) wherein: 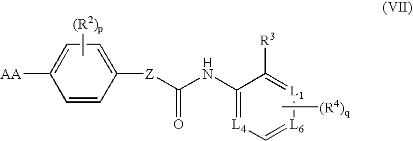
AA is independently selected from halo or triflate; and
Z, R², R³, R⁴, L, p and q are as defined in claim 1.

9. A process for the manufacture of a compound of formula II, or a pharmaceutically acceptable salt or an in vivo hydrolysable ester or amide thereof, according to claim 1, which process comprises the steps of:
a) reacting the following compounds in the presence of a catalyst:
iv) a nucleophile of formula (III) or (IV) wherein the nucleophilic group is either W or is located within R¹;
v) allene gas or a substituted allene; and
vi) a halogen or triflate substituted aryl molecule of formula (IX) 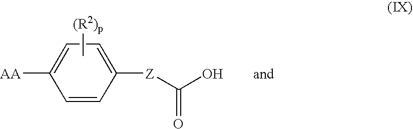
b) reacting the product of (a) with a compound of formula (X) in the presence of a coupling reagent 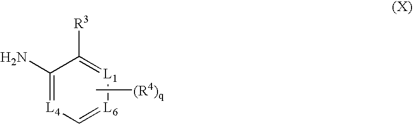
wherein AA is independently selected from halo or triflate; and
Z, R², R³, R⁴, L, p and q are as defined in claim 1.

10. A process as claimed in claim 8 or 9 wherein the catalyst is a palladium catalyst.

11. A process as claimed in claim 8 or 9 wherein AA is bromide or iodide.

12. A process as claimed in claim 8 or 9 wherein the coupling reagent is 4-(4,6-Dimethoxy-1,3,5-triazin-1-yl)-4-methyl-morpholinium chloride.

13. A process as claimed in claim 8 or 9 wherein the process is carried out on a polymer support.

14. A process for the manufacture of a compound of formula (II) or a pharmaceutically acceptable salt or an in vivo hydrolysable ester or amide thereof, according to claim 1, which process comprises cleaving a compound of formula (VIII); 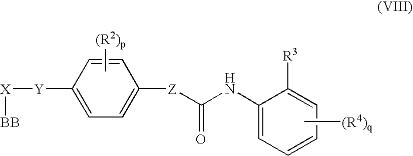
wherein X, Y, Z, R², R³, R⁴, p and q are as defined in claim 1; and BB is a solid phase resin.

15. A process as claimed in claim 14 wherein the resin possesses an —NH— or —NH₂— moiety.

16. A process as claimed in claim 15 wherein the resin is Rink Amide MBHA resin.

17. A compound of the formula II, or a pharmaceutically acceptable salt or an in vivo hydrolysable ester or amide thereof, as claimed in any of claims 1 to 6 for use as a medicament.

18. A method of treatment or alleviation of a cellular proliferative and/or differentiative disorder which comprises administering a therapeutically effective amount of compound of formula (II), or a pharmaceutically acceptable salt or an in vivo hydrolysable ester or amide thereof, as claimed in any of claims 1 to 6 to a patient suffering from such a disorder.

19. The use of a compound of formula (II) in the manufacture of a medicament for the treatment of a cellular proliferative and/or differentiative disorder.

20. The method according to claim 18 or the use according to claim 19 wherein the cellular proliferative and/or differentiative disorder is cancer.

21. The method or use according to claim 20 wherein the cancer is selected from the group consisting of carcinoma, sarcoma, metastatic disorders or hematopoietic neoplastic disorders.

22. The method or use according to claim 20 wherein the cancer is selected from the group consisting of leukaemia, non-small cell lung cancers, colonic cancers, breast cancers, ovarian cancers, renal cancers and melanoma.
